(19) United States
(12) Patent Application Publication
(10) Pub. No.: US 2026/0337259 A1
(43) Pub. Date: Oct. 29, 2026

(54) Targeting brown adipogenesis to prevent cardiotoxicity of chemotherapy

(71) Applicant: Washington University, St. Louis, MO (US)

(72) Inventors: Ali Javaheri, St. Louis, MO (US); Mualla Ozcan, St. Louis, MO (US)

(73) Assignee: Washington University, St. Louis, MO (US)

(21) Appl. No.: 19/662,952

(22) Filed: Apr. 29, 2026

Related U.S. Application Data
(60) Provisional application No. 63/796,375, filed on Apr. 29, 2025.

(51) Int. Cl. C07K 16/18; A61K 31/704; A61K 39/00; A61P 9/00

(52) U.S. Cl. CPC C07K 16/18; A61K 31/704; A61P 9/00; A61K 2039/505; A61K 2039/545; C07K 2317/76

(57) ABSTRACT

A method for preventing doxorubicin-induced cardiotoxicity is disclosed. The methods include administering a therapeutically effective amount of an inhibitor to brown adipose tissue signaling and expansion, including a SLIT2 inhibitor, as an adjuvant to the doxorubicin treatment. In one aspect, the method for preventing doxorubicin-induced cardiotoxicity includes administering a therapeutic amount of an anti-SLIT2 antibody.

### CROSS-REFERENCE TO RELATED APPLICATIONS

This application claims the benefit of priority to U.S. Provisional Application Ser. No. 63/796,375 filed on Apr. 29, 2025, which is incorporated herein by reference in its entirety.

### STATEMENT REGARDING FEDERALLY SPONSORED RESEARCH OR DEVELOPMENT

This invention was made with government support under HL155344 awarded by the National Institutes of Health (NIH). The government has certain rights in the invention.

### MATERIAL INCORPORATED-BY-REFERENCE

Not applicable.

### FIELD OF THE DISCLOSURE

The present disclosure generally relates to methods for the prevention of cardiotoxicity associated with chemotherapy.

### BACKGROUND OF THE DISCLOSURE

Anthracyclines (doxorubicin or Dox) are among the most commonly used chemotherapeutic agents. However, anthracyclines also cause cardiotoxicity and heart failure, leading to an increased risk of mortality and decreased quality of life among cancer survivors. Dox causes skeletal and cardiac muscle wasting but the exact mechanisms underlying Dox cardiotoxicity are complex and multifactorial.

Brown adipose tissue (BAT), a specialized type of adipose tissue, was previously identified as a thermogenic organ that dissipates chemical energy as heat. The key regulator of BAT thermogenesis is mitochondrial uncoupling protein-1 (UCP1), which uncouples respiration from ATP synthesis and increases thermogenesis. SLIT2, a member of the family of Slit homolog proteins has recently been described as a regulator of UCP1-induced thermogenesis. Activation of brown adipogenesis can improve obesity, diabetes mellitus, and lipid metabolism, based on its effects on whole-body energy homeostasis or paracrine effects. However, controversy exists regarding the role of brown adipogenesis in cachexia and muscle wasting. On one hand, thermogenic fat contributes to cancer cachexia, on the other hand, brown fat can induce muscle growth. Additionally, while some studies showed BAT activation with Dox both in humans and mice, the role of BAT in Dox cardiomyopathy is unknown.

The popular fasting strategy, intermittent fasting (IF), has emerged as a potential activator of white adipose browning or beiging, and hence thermogenesis. IF improves cardiovascular risk factors in multiple observational studies, and this can potentially be through the activation of thermogenesis. However, recent studies have emerged as to whether IF can promote cardiovascular events. In the setting of Dox chemotherapy, it has recently been found that ADF, a type of IF with 24 h of fasting alternating with 24 h of feeding, promotes mortality, cachexia, and cardiotoxicity in mice, one of the first mechanistic studies to describe the toxic effects of fasting on myocardial function.

### SUMMARY OF THE DISCLOSURE

Among the various aspects of the present disclosure is the provision of a method to treat chemotherapy-induced cardiotoxicity. The method comprises administering an inhibitor configured to inhibit the signaling or expansion of brown adipose tissue (BAT).

In some aspects, the inhibitor may target a protein or a gene in the SLIT2 signaling pathway. In some aspects, the inhibitor targeting the SLIT2 signaling pathway may be an inhibitor comprised of UCP1, SLIT2, ROBO1, ROBO2, ROBO3, ROBO4, mTOR, TFEB or any combination thereof. In some aspects, the inhibitor is an anti-SLIT2 antibody. In some aspects, the anti-SLIT2 antibody is administered in at least two doses. In some aspects, the anti-SLIT2 antibody is administered in 6 doses. In some aspects, the therapeutically effective amount of the anti-SLIT2 antibody is 0.3 mg/kg. In some aspects, treating the chemotherapy-induced cardiotoxicity comprises maintaining a baseline percentage of brown adipose tissue, the baseline percentage of brown adipose tissue comprising the percentage of the subject before the chemotherapy. In some aspects, the baseline percentage of brown adipose tissue may be the percentage of brown adipose tissue present in a subject before fasting or administration of a chemotherapy agent. In some aspects, the chemotherapeutic is doxorubicin. In some aspects, treating the chemotherapy-induced cardiotoxicity comprises the subject maintaining a non-fasting diet.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A is a schematic diagram of an experimental protocol in which chow-fed C57BL/6 mice (n=30) are treated with Dox (5 mg/kg i.p.×3 doses) and randomized to adlib or 2 weeks post-Dox ADF, followed by echocardiography before euthanasia. All echocardiography was performed on a fed day.

FIG. 1B is a pair of representative images of 2-dimensional-directed M-mode echocardiographic images of mice treated with Adlib Dox and ADF Dox.

FIG. 1C is a graph of echocardiographic left ventricular (LV) mass/tibia length in mice treated with Adlib Dox and ADF Dox (n=15/group; Student's t-test). Data were obtained by quantification of blinded echocardiographic assessment (under 2% Avertin, IP) of 2-dimensional images. Mice were sacrificed on a fed day. Data are mean±SEM.

FIG. 1D is a graph of ejection fraction in mice treated with Adlib Dox and ADF Dox (n=15/group; Student's t-test). Data were obtained by quantification of blinded echocardiographic assessment (under 2% Avertin, IP) of 2-dimensional images. Mice were sacrificed on a fed day. Data are mean±SEM. treated with Adlib Dox and ADF Dox (n=15/group; Student's t-test).

FIG. 1E is a graph of end diastolic volume/body weight in mice treated with Adlib Dox and ADF Dox (n=15/group; Student's t-test). Data were obtained by quantification of blinded echocardiographic assessment (under 2% Avertin, IP) of 2-dimensional images. Mice were sacrificed on a fed day. Data are mean±SEM.

FIG. 1F is a graph of heart rate in mice treated with Adlib Dox and ADF Dox (n=15/group; Student's t-test). Data were obtained by quantification of blinded echocardiographic assessment (under 2% Avertin, IP) of 2-dimensional images. Mice were sacrificed on a fed day. Data are mean±SEM.

FIG. 1G is a set of representative images of mid-myocardial sections from mice treated with Dox (5 mg/kg i.p.×3 doses) and randomized to adlib or 2 weeks post-Dox ADF stained with picrosirius red/fast green (4× scale bar=500 μm, 20× scale bar=100 μm).

FIG. 1H is a graph of blinded quantification of fibrosis based on images similar to those of FIG. 1G (n=5/group; Student's t-test).

FIG. 2A is a graph of percent change in body weight of chow-fed C57BL/6 mice (n=50) treated with Dox (5 mg/kg i.p.×3 doses) and randomized to adlib or 2 weeks post-Dox ADF, followed by body composition analysis before euthanasia. % change in body weight at euthanasia vs. baseline is shown (n=25/group, Student's t-test); data are mean±SEM.

FIG. 2B is a graph of lean mass % change at euthanasia vs. baseline in a subset of mice treated with Adlib Dox and ADF Dox, measured on a fed day by EchoMRI (n=10/group, Student's t-test); data are mean±SEM.

FIG. 2C is a graph of body fat mass % change at euthanasia vs. baseline in a subset of mice treated with Adlib Dox and ADF Dox, measured on a fed day by EchoMRI (n=10/group, Student's t-test); data are mean±SEM.

FIG. 2D is a graph of calf muscle (gastrocnemius and soleus) weight/body weight in mice treated with Adlib Dox and ADF Dox (n=25/group, Student's t-test); data are mean±SEM.

FIG. 2E is a graph of tibialis anterior muscle weight/body weight in mice treated with Adlib Dox and ADF Dox (n=25/group, Student's t-test); data are mean±SEM.

FIG. 2F is a graph of heart weight/tibia length in mice treated with Adlib Dox and ADF Dox (n=25/group, Student's t-test); data are mean±SEM.

FIG. 2G is a graph of liver weight/body weight in mice treated with Adlib Dox and ADF Dox (n=25/group, Student's t-test); data are mean±SEM.

FIG. 3A is a graph of interscapular BAT/body weight of chow-fed C57BL/6 mice (n=20) treated with Dox (5 mg/kg i.p.×3 doses) and randomized to adlib or 2 weeks post-Dox ADF Dox, followed by body composition analysis before euthanasia; (n=10/group, Student's t-test); data are mean±SEM.

FIG. 3B is a graph of inguinal white adipose tissue/body weight in mice treated with Adlib Dox and ADF Dox (n=10/group, Student's t-test); data are mean±SEM.

FIG. 3C is a table summarizing adipose-related pathways regulated in Dox+ADF vs Dox mice based on aptamer-based plasma proteomics (−log[p] values of Fisher exact test and z-scores are shown).

FIG. 3D is a graph of plasma SLIT2 levels in mice treated with Adlib Dox and ADF Dox based on aptamer-based measurements (n=6/group, Student's t-test); data are mean±SEM.

FIG. 3E is a graph of plasma SLIT2 levels in subjects with Dox cardiomyopathy vs. other cardiomyopathies, based on aptamer-based measurements, adjusted for covariates (n=47-2167/group, false discovery rate <0.0001); data are mean±SEM.

FIG. 3F is a graph of body mass index in all subjects from FIG. 3E, stratified based on plasma SLIT2 levels (n=743-747/tertile, one-way ANOVA); data are mean±SEM.

FIG. 3G is a graph of NYHA classification in all subjects from FIG. 3E, stratified based on plasma SLIT2 levels (n=743-747/tertile, one-way ANOVA); data are mean±SEM.

FIG. 3H contains representative images of immunoblots of SLIT2 in interscapular brown adipose protein lysates from mice treated with Adlib Dox and ADF Dox. β-tubulin serves as a loading control.

FIG. 3I is a graph quantifying SLIT2 relative to β-tubulin based on immunoblot images similar to those of FIG. 3H (n=8/group, Mann-Whitney test); data are mean±SEM.

FIG. 3J contains representative images of immunoblots of SLIT2 in inguinal white adipose protein lysates from mice treated with Adlib Dox and ADF Dox. β-tubulin serves as a loading control.

FIG. 3K is a graph quantifying SLIT2 relative to β-tubulin from immunoblot images similar to those of FIG. 3J (n=5/group, Mann-Whitney test); data are mean±SEM.

FIG. 3L is a set of representative images of H&E staining of inguinal white adipose tissue sections from mice treated with Adlib saline, Adlib Dox, and ADF post-Dox at 10× and 20× magnification.

FIG. 3M is a set of representative images of H&E staining of interscapular brown adipose tissue sections from mice treated with Adlib saline, Adlib Dox, and ADF post-Dox at 20× and 40× magnification.

FIG. 4A is a schematic diagram summarizing a timeline for chow-fed C57BL/6 mice receiving Dox (5 mg/kg i.p., 3 doses) plus vehicle or recombinant SLIT2 (rSLIT2) (0.3 mg/kg i.p.×5 doses) (n=7/group), and undergoing echocardiography before euthanasia in an experiment described herein.

FIG. 4B is a graph of % change in body weight in mice receiving vehicle, Dox, or Dox+rSLIT2 (n=7/group, one-way ANOVA with Sidak's correction for multiple comparisons).

FIG. 4C is a graph of interscapular brown adipose tissue/body weight in mice receiving vehicle, Dox, or Dox+rSLIT2 (n=7/group, one-way ANOVA with Sidak's correction for multiple comparisons).

FIG. 4D is a graph of inguinal white adipose tissue/body weight in mice receiving vehicle, Dox, or Dox+rSLIT2 (n=7/group, one-way ANOVA with Sidak's correction for multiple comparisons).

FIG. 4E is a graph of calf muscle (gastrocnemius and soleus) weight/tibia length in mice receiving vehicle, Dox, or Dox+rSLIT2 (n=7/group, one-way ANOVA with Sidak's correction for multiple comparisons).

FIG. 4F is a graph of heart weight/tibia length in mice receiving vehicle, Dox, or Dox+rSLIT2 (n=7/group, one-way ANOVA with Sidak's correction for multiple comparisons).

FIG. 4G is a set of representative 2-dimensional-directed M-mode echocardiographic images in mice receiving vehicle, Dox, or Dox+rSLIT2.

FIG. 4H is a graph of left ventricular ejection fractions in mice receiving vehicle, Dox, or Dox+rSLIT2 (n=7/group, one-way ANOVA with Sidak's correction for multiple comparisons). Data were obtained by quantification of blinded echocardiographic assessment (under 2% Avertin, IP) of 2-dimensional images.

FIG. 4I is a graph of echocardiographic left ventricular (LV) mass/tibia lengths in mice from (A) (n=7/group, one-way ANOVA with Sidak's correction for multiple comparisons). Data were obtained by quantification of blinded echocardiographic assessment (under 2% Avertin, IP) of 2-dimensional images.

FIG. 4J is a pair of representative images of mid-myocardial sections from mice receiving vehicle, Dox, or Dox+rSLIT2 stained with Picrosirius red/fast green (scale bar=100 μm).

FIG. 4K is a graph quantifying fibrosis form images similar to those of FIG. 4J (n=5/group; Student's t-test).

FIG. 5A is a schematic diagram defining a timeline of an experimental protocol showing chow-fed C57BL/6 mice (n=16) treated with Dox (5 mg/kg i.p.×3 doses) and randomized to adlib or 2 weeks post-Dox ADF and IgG vs. anti-SLIT2 (0.3 mg/kg×6 doses), with echocardiography at baseline and before euthanasia, as shown in the schematic.

FIG. 5B is a set of representative 2-dimensional-directed M-mode echocardiographic images in mice treated with Dox IgG, Dox ADF IgG, or Dox ADF anti-SLIT2.

FIG. 5C is a graph of echocardiographic left ventricular (LV) mass change at euthanasia vs baseline in mice treated with Dox IgG, Dox ADF IgG, or Dox ADF anti-SLIT2 (n=4-6/group; one-way ANOVA with Sidak's correction for multiple comparisons).

FIG. 5D is a graph of left ventricular ejection fraction change at euthanasia vs baseline in mice treated with Dox IgG, Dox ADF IgG, or Dox ADF anti-SLIT2 (n=4-6/group; one-way ANOVA with Sidak's correction for multiple comparisons).

FIG. 5E is a graph of end diastolic volume change at euthanasia vs baseline in mice treated with Dox IgG, Dox ADF IgG, or Dox ADF anti-SLIT2 (n=4-6/group; one-way ANOVA with Sidak's correction for multiple comparisons).

FIG. 5F is a set of representative images of mid-myocardial sections from mice treated with Dox (5 mg/kg i.p.×3 doses) and randomized to adlib or 2 weeks post-Dox ADF stained with picrosirius red/fast green (scale bar=100 μm).

FIG. 5G is a graph of blinded quantification of fibrosis based on images similar to those of FIG. 5F (n=3-6/group; one-way ANOVA with Sidak's correction for multiple comparisons).

FIG. 5H is a set of representative images of mid-myocardial sections from mice treated with Dox (5 mg/kg i.p.×3 doses) and randomized to adlib or 2 weeks post-Dox ADF stained with wheat germ agglutinin (WGA) (scale bar=50 μm).

FIG. 5I is a graph of blinded cell size quantification based on images similar to those of FIG. 5H (n=3-6/group; one-way ANOVA with Sidak's correction for multiple comparisons).

FIG. 6A contains representative immunoblot images for p-mTORSer2448/mTOR; β-tubulin serves as a loading control. Chow-fed C57BL/6 mice were treated with Dox (5 mg/kg i.p.×3 doses) and randomized to adlib (Adlib DOX) or 2 weeks post-Dox ADF (ADF Dox).

FIG. 6B is a graph quantifying p-mTORSer2448/mTOR based on immunoblot images similar to those of FIG. 6A (n=8/group, Student's t-test).

FIG. 6C contains representative immunoblot images for UCP1 in brown adipose lysates from mice treated with Adlib Dox or ADF Dox, β-tubulin serves as a loading control.

FIG. 6D is a graph quantifying UCP1 based on immunoblot images similar to those of FIG. 6C (n=7/group, Student's t-test).

FIG. 6E contains representative immunoblot images for p-mTORSer2448/mTOR are shown, β-tubulin serves as a loading control. Inguinal white adipose tissue protein lysates were prepared from mice treated with Adlib Dox or ADF Dox.

FIG. 6F is a graph quantifying p-mTORSer2448/mTOR based on immunoblot images similar to those of FIG. 6E (n=5/group, Student's t-test).

FIG. 6G contains representative immunoblot images for UCP1 in inguinal white adipose lysates from mice treated with Adlib Dox or ADF Dox, β-tubulin serves as a loading control.

FIG. 6H is a graph quantifying UCP1 based on immunoblot images similar to those of FIG. 6G (n=10/group, Mann-Whitney test).

FIG. 6I is a graph of body weight % change. Chow-fed WT and UCP1KO mice were treated with Dox (5 mg/kg i.p.×3 doses), underwent echocardiography before Dox and before euthanasia (Mann-Whitney test, n=6-9/group).

FIG. 6J is a graph of heart weight/tibia length in WT and UCP1KO mice (Mann-Whitney test, n=6-9/group).

FIG. 6K is a representative set of 2-dimensional-directed M-mode echocardiographic images in WT and UCP1KO mice

FIG. 6L is a graph showing Echocardiographic left ventricular (LV) mass at euthanasia vs. baseline in WT and UPC1KO mice (n=6-9/group; two-way ANOVA with Sidak's correction for multiple comparisons).

FIG. 6M is a graph of left ventricular ejection fraction in WT and UCP1KO mice (n=6-9/group; two-way ANOVA with Sidak's correction for multiple comparisons).

FIG. 7 is a schematic diagram of an experimental protocol showing mouse and human treatment timelines and outcomes.

FIG. 8A is a representative set of 2-dimensional-directed M-mode echocardiographic images. Chow-fed C57BL/6 mice (n=5-6/group) were treated with saline (5 mg/kg i.p.×3 doses) and randomized to adlib or 2 weeks post-saline ADF, followed by echocardiography before euthanasia.

FIG. 8B is a graph of left ventricular ejection fraction in mice treated with saline (adlib) or ADF (n=5-6/group; Student's t-test).

FIG. 8C is a graph of echocardiographic left ventricular (LV) mass/tibia length in mice treated with saline (adlib) or ADF (n=5-6/group; Student's t-test).

FIG. 8D is a graph of end diastolic volume/body weight in mice treated with saline (adlib) or ADF (n=5-6/group; Student's t-test).

FIG. 9A is a graph of % change in body weight at euthanasia vs. baseline. Chow-fed C57BL/6 mice (n=5-6/group) were treated with saline (5 mg/kg i.p.×3 doses) and randomized to adlib or 2 weeks post-saline ADF, followed by echocardiography before euthanasia (n=5-6/group, Student's t-test).

FIG. 9B is a graph of heart weight/tibia length in mice treated with saline (Adlib) or ADF (n=5-6/group, Student's t-test).

FIG. 9C is a graph of calf muscle (gastrocnemius and soleus) weight/body weight in mice treated with saline (Adlib) or ADF (n=5-6/group, Student's t-test).

FIG. 9D is a graph of interscapular brown adipose tissue/body weight in mice treated with saline (Adlib) or ADF (n=5-6/group, Student's t-test).

FIG. 9E is a graph of inguinal white adipose tissue/body weight in mice treated with saline (Adlib) or ADF (n=5-6/group, Student's t-test).

FIG. 10A is a graph of % change in body weight at euthanasia. Chow-fed C57BL/6 mice (n=30) were treated with Dox (5 mg/kg i.p.×3 doses) and randomized to adlib or 2 weeks post-Dox ADF, followed by body composition analysis before euthanasia (n=15/group, Student's t-test).

FIG. 10B is a graph of lean mass in mice treated with Dox (adlib) or ADF (n=15/group, Student's t-test).

FIG. 10C is a graph of fat mass in mice treated with Dox (adlib) or ADF (n=15/group, Student's t-test).

FIG. 11A is a schematic diagram illustrating an experimental protocol in which mice are treated with doxorubicin (4×5 mg/kg i.p.) and given adlib or sustained ADF, followed by echocardiography and echoMRI over 5 weeks.

FIG. 11B contains immunoblot images of TFEB and controls H3 and GAPDH proteins in donor, DC, and NICM tissues.

FIG. 11C is a graph quantifying nuclear TFEB content relative to H3 levels based on immunoblot images similar to those of FIG. 11B.

FIG. 11D is a survival curve of mice treated with combinations of adlib, saline, dox, and ADF.

FIG. 11E is a graph of calf muscles/tibia length in mice treated with combinations of adlib, saline, dox, and ADF.

FIG. 11F is a graph of LV mass/tibia length in mice treated with combinations of adlib, saline, dox, and ADF.

FIG. 11G is a graph of nuclear TFEB content relative to H3 in mice treated with combinations of adlib, saline, dox, and ADF.

FIG. 11H is a graph of MuRF1 level relative to beta-tubulin in mice treated with combinations of adlib, saline, dox, and ADF.

FIG. 11I is a survival curve of AAV9-CMV-null and AAV9-CMV-TFEB mice treated with Dox or saline (n=5).

FIG. 11J is a graph of percent ejection fraction in AAV9-CMV-null and AAV9-CMV-TFEB mice.

FIG. 11K is a graph of LV mass in AAV9-CMV-null and AAV9-CMV-TFEB mice.

FIG. 11L is a graph of MuRf1 level post Dox relative to beta-tubulin levels in TFEB-floxed and TFEB-CMKO mice.

FIG. 11M is a graph of GDF15 levels in in AAV9-CMV-null and AAV9-CMV-TFEB mice treated with saline or dox.

FIG. 11N is a graph of LV mass/tibia length in control or GDF15 mice.

FIG. 11O is a scatter plot of stroke volume relative to LV mass.

### DETAILED DESCRIPTION OF THE INVENTION

Anthracycline chemotherapeutics, including doxorubicin, are commonly used to treat many cancers but they frequently cause cardiotoxicity. As described in the examples herein, an accelerated model of doxorubicin-induced cardiotoxicity (DoxTox) in mice was developed that was characterized by BAT expansion and increased SLIT2, a secreted extra-cellular matrix protein known for its role in axon guidance, regulating thermogenesis, and metabolic function.

The present disclosure is based on the discovery that inhibition of SLIT2 and brown adipose tissue (BAT) signaling pathways attenuates doxorubicin-induced cardiotoxicity. The accelerated model of doxorubicin-induced cardiotoxicity (DoxTox) in mice demonstrates an expansion of brown adipose tissue and an increase of SLIT2, which was found to be consistent with plasma proteomic data from patients with Dox-induced cardiomyopathy compared to other heart failure. Moreover, mice subjected to recombinant SLIT2 exhibited an accelerated DoxTox, thus identifying SLIT2 as a causal factor for increased toxicity. These findings demonstrated a role for BAT thermogenesis and SLIT2 in Dox-induced toxicity.

The effect of fasting initiated after Dox chemotherapy was tested and found that fasting caused a decline in lean body mass and left ventricular mass index, but increased body fat content, liver weight, and brown adipose tissue (BAT) weight index vs Dox-treated mice not subjected to fasting. Dox+alternate day fasting (ADF) induced SLIT2, which regulates thermogenesis and metabolic function in BAT. Dox plus recombinant SLIT2 decreased left ventricular ejection fraction vs Dox alone. Plasma proteomics from human patients showed that SLIT2 was the top protein associated with Dox cardiomyopathy vs other non-ischemic cardiomyopathy. This study suggests that SLIT2, an activator of brown adipogenesis, is a driver of Dox cardiotoxicity. Disclosed herein is a method of mitigating Dox-induced cardiotoxicity and improving patient outcomes through the inhibition of SLIT2 as an adjuvant to Dox chemotherapy.

In various aspects, methods of treatment are disclosed to attenuate chemotherapy-induced cardiotoxicity symptoms including, but not limited to, decreased left ventricular ejection fraction, decreased left ventricular mass, increased end-diastolic volume (EDV), loss of cardiac and skeletal muscle mass, increased fat mass, and increased brown adipose mass. In various aspects, methods to treat chemotherapy-induced cardiotoxicity comprise the administration of a therapeutically effective amount of brown adipose tissue (BAT) signaling inhibitor. In various aspects, a BAT signaling inhibitor can be any compound or molecule that inhibits upstream or downstream signaling of BAT, including, but not limited to, UCP1, SLIT2, ROBO, mTOR inhibitors, and any combination thereof. In some aspects, the inhibitor may comprise an antibody, a recombinant protein, a siRNA, and gene knockdown or silencing.

As described in the examples herein, exogenous administration of a SLIT2 inhibitor attenuated doxorubicin-induced cardiotoxicity. Administration of a SLIT2 inhibitor attenuated changes in left ventricular ejection fraction, left ventricular mass, EDV, and fractional shortening.

The inhibitor may be administered as a single dose or multiple doses. The inhibitor may be administered in 6 doses. The inhibitor may be administered before administration of a chemotherapeutic, in conjunction with a chemotherapeutic, or after treatment with a chemotherapeutic.

The inhibitor may be an antibody. The inhibitor may be an anti-SLIT2, anti-UCP1, anti-ROBO1, anti-ROBO2, anti-ROBO3, anti-ROBO4, or anti-mTOR antibody. The anti-SLIT2 antibody may be administered at a dose of 0.3 mg/kg.

Treating chemotherapy-induced cardiotoxicity may comprise a subject maintaining a normal diet, a non-intermittent fasting (IF) diet, or a non-fasting diet. IF may be avoided before, during, or after treatment with a chemotherapeutic.

### Therapeutic Methods

Also provided is a process of treating, preventing, or reversing doxorubicin-induced cardiotoxicity in a subject in need by administration of a therapeutically effective amount of a SLIT2 inhibitor or a BAT signaling inhibitor, so as to prevent, reduce, or reverse the doxorubicin-induced cardiotoxicity as characterized by increased brown adipose mass, loss of cardiac and skeletal muscle, and/or cardiac function of a patient.

Methods described herein are generally performed on a subject in need thereof. A subject in need of the therapeutic methods described herein can be a subject having, diagnosed with, suspected of having, or at risk for developing chemotherapy-associated cardiotoxicity. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of SLIT2 inhibitor or BAT signaling inhibitor is, for example, an amount that would cause the desired therapeutic effect in a subject while minimizing undesired side effects. In various embodiments, an effective amount of SLIT2 inhibitor described herein can substantially inhibit SLIT2 signaling, ROBO signaling, UCP1, and brown adipose tissue signaling to attenuate doxorubicin-induced cardiotoxicity.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of SLIT2 inhibitor or BAT signaling inhibitor can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to prevent, reduce, or reverse doxorubicin-induced cardiotoxicity.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single-dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of SLIT2 inhibitor or BAT signaling inhibitor can occur as a single event or over a time course of treatment. For example, a SLIT2 inhibitor or BAT signaling inhibitor can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accordance with the methods described herein can be performed prior to, concurrent with, or after conventional treatment modalities for the prevention, reduction, or reversal of chemotherapy-associated cardiotoxicity.

A SLIT2 inhibitor or BAT signaling inhibitor can be administered simultaneously or sequentially with another agent, such as an antibiotic, an anti-inflammatory, or another agent. For example, a SLIT2 inhibitor or BAT signaling inhibitor can be administered simultaneously with another agent, such as an antibiotic or an anti-inflammatory. Simultaneous administration can occur through the administration of separate compositions, each containing one or more of a SLIT2 inhibitor or BAT signaling inhibitor, an antibiotic, an anti-inflammatory, or another agent. Simultaneous administration can occur through the administration of one composition containing two or more of a SLIT2 inhibitor or BAT signaling inhibitor, an antibiotic, an anti-inflammatory, or another agent. A SLIT2 inhibitor or BAT signaling inhibitor can be administered sequentially with an antibiotic, an anti-inflammatory, or another agent. For example, a SLIT2 inhibitor or BAT signaling inhibitor can be administered before or after the administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10:0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: Chem Bridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about-2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition as described herein and, in certain embodiments, instructions for administration. Such kits can facilitate the performance of the methods described herein. When supplied as a kit, the different components of the composition can be packaged in separate containers and admixed immediately before use. Components include, but are not limited to compositions containing a SLIT2 inhibitor or BAT signaling inhibitor as described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: SLIT2 is a Driver of Doxorubicin Cardiotoxicity Through Adipose-Cardiac Crosstalk

Following a cardiac insult, heart failure can escalate into multi-system organ failure, though the specifics of inter-organ communication in this progression are not fully understood. The commonly used chemotherapeutic agent doxorubicin (Dox) initially induces cardiac muscle wasting, leading to heart failure in a subset of patients. This study has shown that alternate-day fasting (ADF) potentiates Dox-induced cardiotoxicity, increases adiposity, and brown adipose mass. Dox+ADF upregulates SLIT2, an activator of thermogenesis, in plasma and adipose tissue, which parallels the observation using aptamer-based proteomics that plasma SLIT2 is the most significant and pronounced protein identifying human Dox cardiomyopathy (vs other cardiomyopathy). Dox+ADF also stimulates the SLIT2-target UCP1 in inguinal adipose tissue, while UCP1 knockout attenuates Dox cardiotoxicity. Further studies show that SLIT2 is both necessary and sufficient for Dox cardiotoxicity. These findings reveal an adipose-cardiac crosstalk mediated by SLIT2 as a mechanism for Dox cardiomyopathy.

Anthracyclines (doxorubicin or Dox) are among the most commonly used chemotherapeutic agents. However, anthracyclines also cause cardiotoxicity and heart failure, leading to an increased risk of mortality and decreased quality of life among cancer survivors. Dox causes skeletal and cardiac muscle wasting but the exact mechanisms underlying Dox cardiotoxicity are complex and multifactorial. There is a tremendous need for innovative, mechanism-based cardioprotective strategies to effectively mitigate Dox cardiomyopathy in patients.

Brown adipose tissue (BAT), a specialized type of adipose tissue, was previously identified as a thermogenic organ that dissipates chemical energy as heat. The key regulator of BAT thermogenesis is mitochondrial uncoupling protein-1 (UCP1), which uncouples respiration from ATP synthesis and increases thermogenesis. SLIT2, a member of the family of Slit homolog proteins best known for its role in axonal guidance, has recently been described as a regulator of UCP1-induced thermogenesis. There are at least two types of thermogenic fat cells: brown fat and beige fat, dispersed through white fat depots. Recent studies have shown that brown fat is functional in adult humans. Activation of brown adipogenesis can improve obesity, diabetes mellitus, and lipid metabolism, based on its effects on whole-body energy homeostasis or paracrine effects. However, controversy exists regarding the role of brown adipogenesis in cachexia and muscle wasting. On one hand, thermogenic fat contributes to cancer cachexia, on the other hand, brown fat can induce muscle growth. Additionally, while some studies showed BAT activation with Dox both in humans and mice, the role of BAT in Dox cardiomyopathy is unknown.

The popular fasting strategy, intermittent fasting (IF), has emerged as a potential activator of white adipose browning or beiging, and hence thermogenesis. IF improves cardiovascular risk factors in multiple observational studies, and this can potentially be through the activation of thermogenesis. However, controversy has recently emerged as to whether IF can promote cardiovascular events. In the setting of Dox chemotherapy, it has recently been found that ADF, a type of IF with 24 h of fasting alternating with 24 h of feeding, promotes mortality, cachexia, and cardiotoxicity in mice, one of the first mechanistic studies to describe the toxic effects of fasting on myocardial function. Notably, the same ADF protocol was shown to rescue murine models of diet-induced obesity, ischemia, and protein-aggregate cardiomyopathy.

Because the majority of published studies have suggested that fasting attenuates Dox cardiotoxicity, this study sought to determine whether ADF would be better tolerated if initiated after Dox chemotherapy. It was found that two weeks of ADF after Dox treatment (Dox+ADF) potentiates Dox cardiotoxicity. In contrast to prior work on sustained ADF, it was found that Dox+ADF promotes adiposity and brown adipose expansion. Using aptamer-based proteomics in mice and humans, it was found that Dox+ADF upregulated SLIT2, a regulator of thermogenesis, which was also identified as the most differentially regulated protein (by p-value and by fold change) in humans with Dox cardiomyopathy (vs other cardiomyopathies). Using recombinant N-terminal SLIT2 and anti-SLIT2 antibodies, this study demonstrated that SLIT2 is necessary and sufficient to drive Dox cardiotoxicity in mice. Further, ADF upregulates SLIT2-target UCP1 in adipose depots, and UCP1 knockout attenuates Dox cardiotoxicity. These studies suggest that an adipose-cardiac axis, driven by SLIT2, accelerates Dox cardiomyopathy and that SLIT2 may be a therapeutic target in this disease setting.

### Results

Two Weeks of Alternate-Day Fasting after Doxorubicin Treatment is Sufficient to Exacerbate Dox Cardiotoxicity

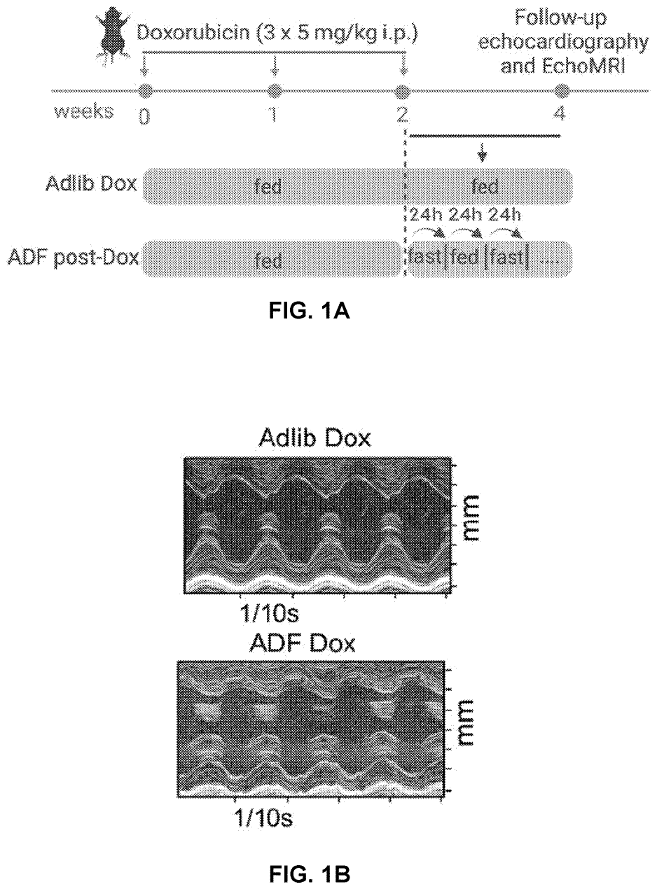

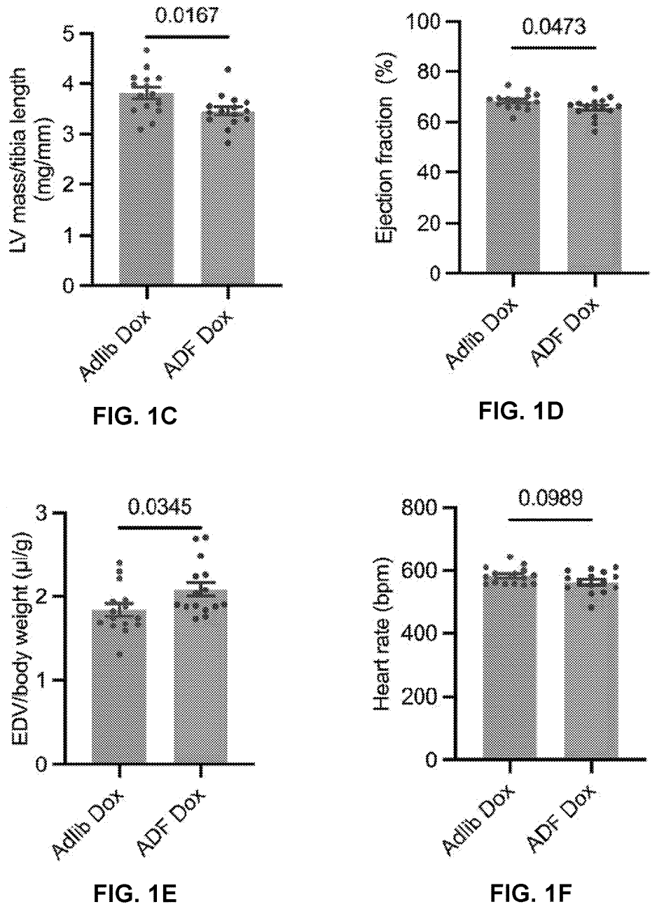

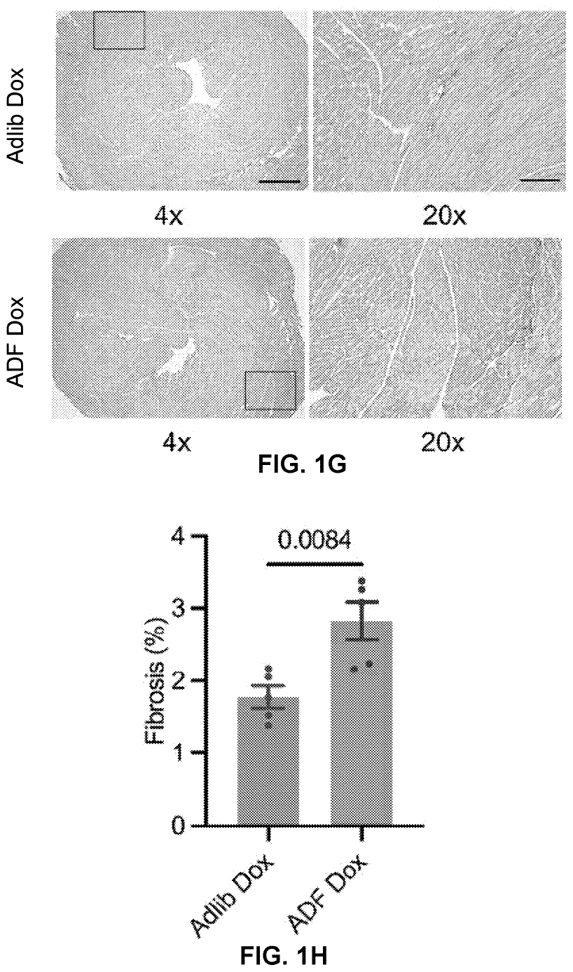

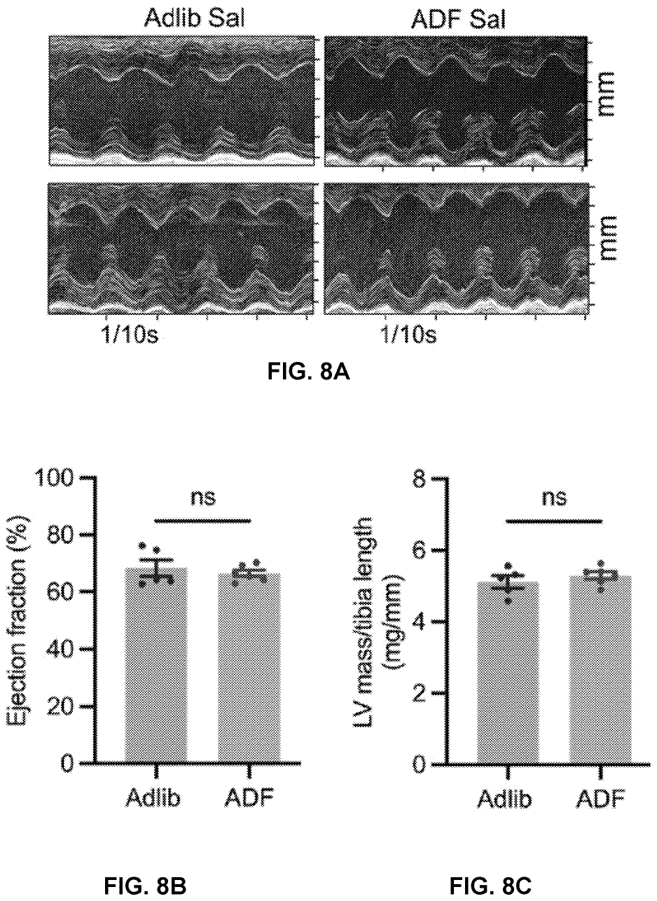

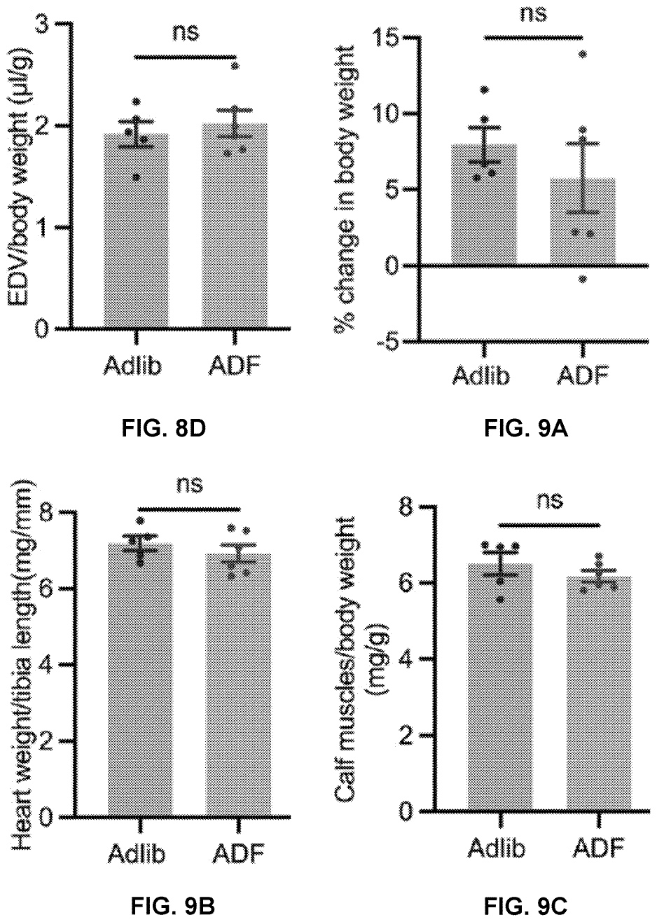

To study ADF initiated after Dox treatment, a protocol was implemented where chow-fed male C57BL/6 mice (n=15/group) were treated with Dox (5 mg/kg intraperitoneally [i.p.] weekly), and following the last Dox injection, randomized to ad libitum feeding or ADF for 2 weeks, as illustrated in FIG. 1A. Because there was significant mortality in prior sustained fasting studies with 4 doses of Dox, in this study, mice received 3 doses of Dox. In control mice injected with saline, 2 weeks of ADF did not demonstrate any significant differences in cardiac parameters compared with ad libitum feeding (FIG. 8A, FIG. 8B, FIG. 8C).

Similar to what was previously observed with sustained ADF, Dox+ADF decreased left ventricular (LV) mass/tibia length (FIG. 1B, FIG. 1C) and left ventricular ejection fraction (LVEF) (FIG. 1D), and increased end-diastolic volume (EDV)/body weight with no changes in heart rate (FIG. 1E, FIG. 1F). Histologic assessment also suggested Dox+ADF worsened cardiotoxicity. The ADF group exhibited increased myocardial fibrosis (FIG. 1G, FIG. 1H) similar to the sustained ADF.

ADF Potentiates Dox Skeletal and Cardiac Muscle Loss while Promoting Adiposity

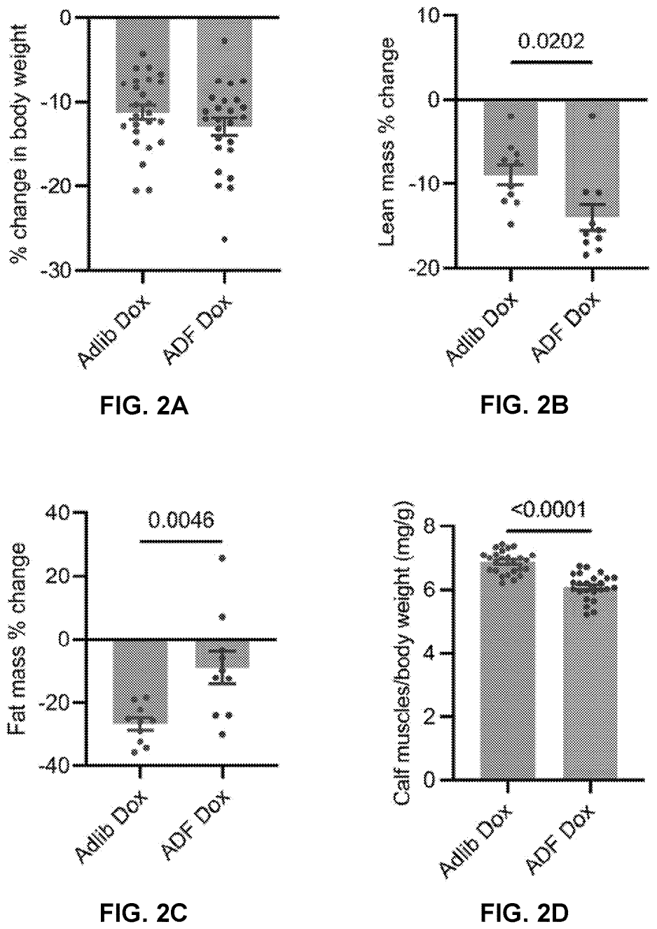

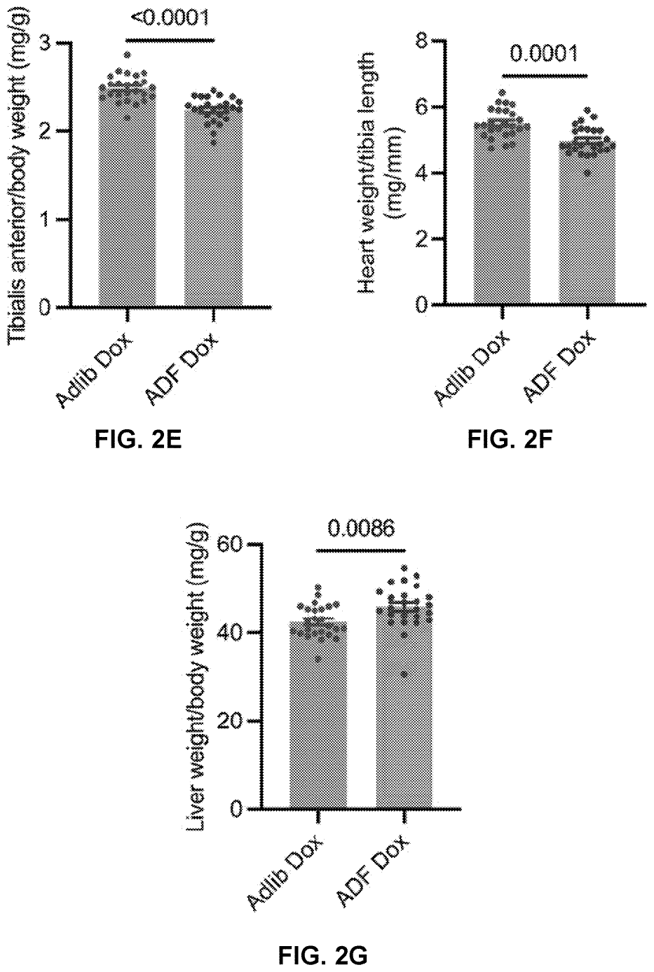

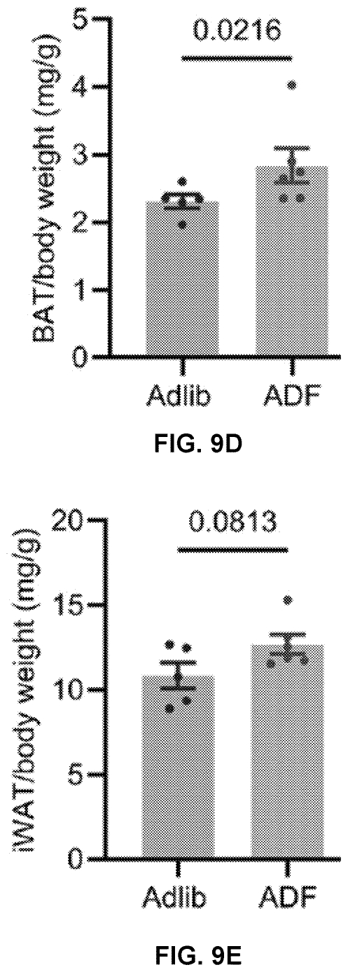

Previously, it has been shown that Dox (vs saline) reduces body weight, skeletal muscle, and fat mass, which was accelerated by sustained fasting. In contrast, 2 weeks of ADF initiated after Dox did not result in significant body weight changes vs Dox alone (FIG. 2A). However, paired EchoMRI-based body composition analysis in a subset of mice, performed on a fed day, revealed that Dox+ADF mice exhibited greater lean (FIG. 2B) vs. fat (FIG. 2C) mass loss, suggesting increased adiposity with ADF. Mice that underwent Dox+ADF showed significant reductions in skeletal muscle and cardiac mass, normalized to body weight and tibia length, respectively, vs. Dox-alone mice (FIG. 2D, FIG. 2E, FIG. 2F). Notably, Dox+ADF mice demonstrated increased liver weight indexed to body weight compared with Dox-alone mice (FIG. 2G), which could potentially be contributing to increased adiposity. In parallel to the echocardiography, ADF did not cause any difference in body weight change and cardiac or skeletal muscle mass (FIG. 9A, FIG. 9B, FIG. 9C).

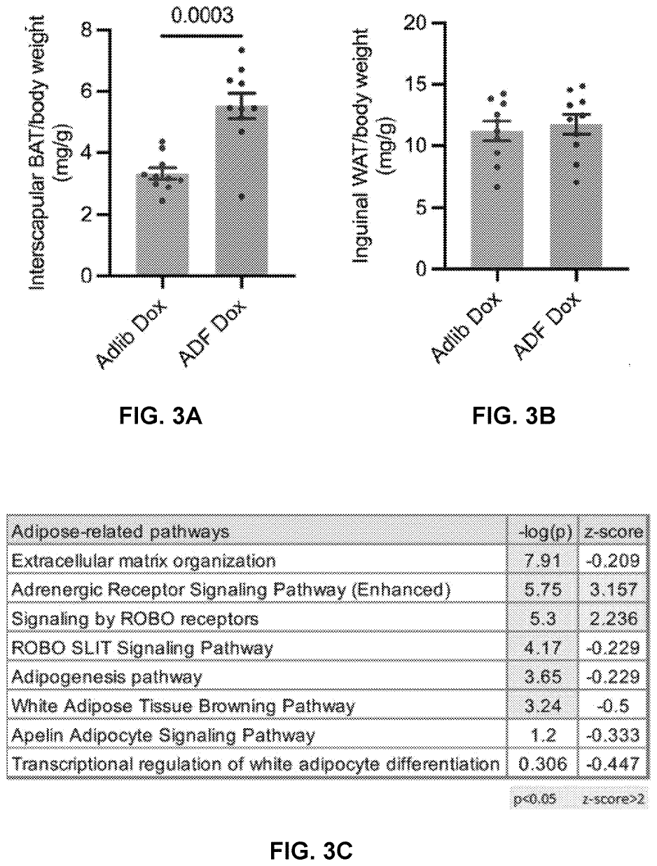

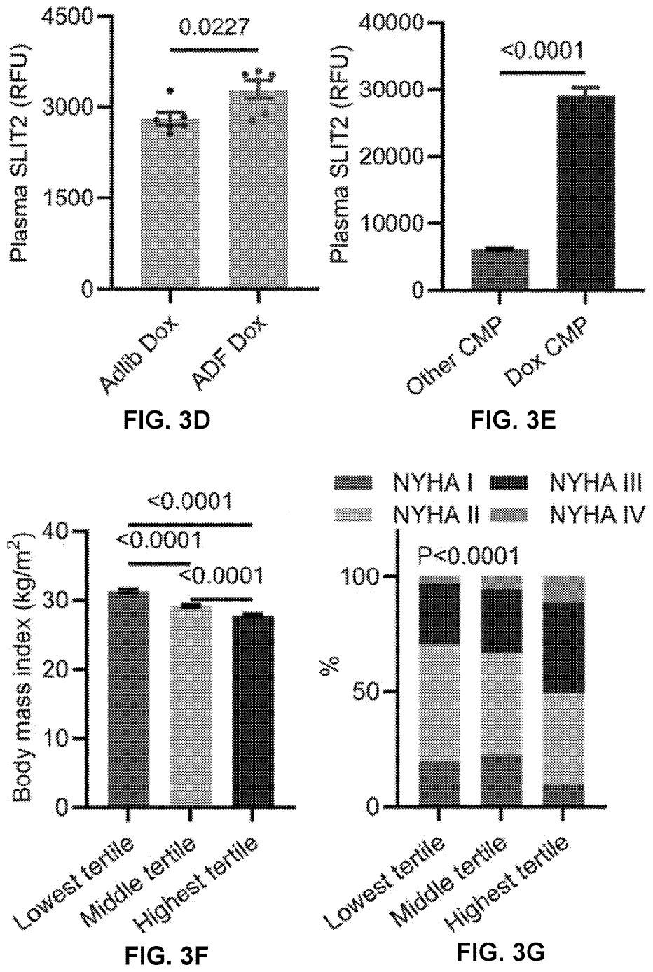

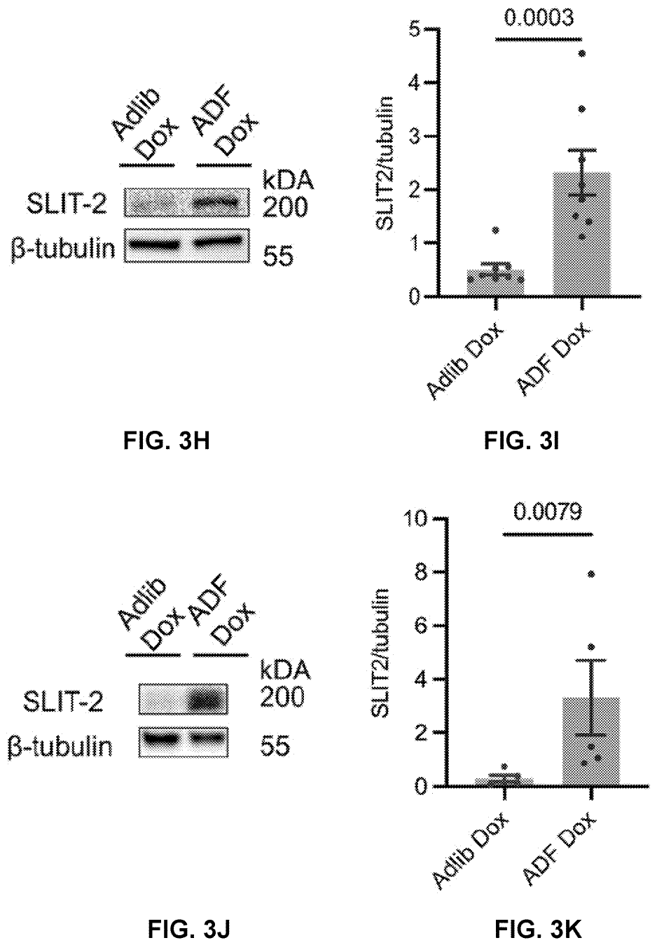

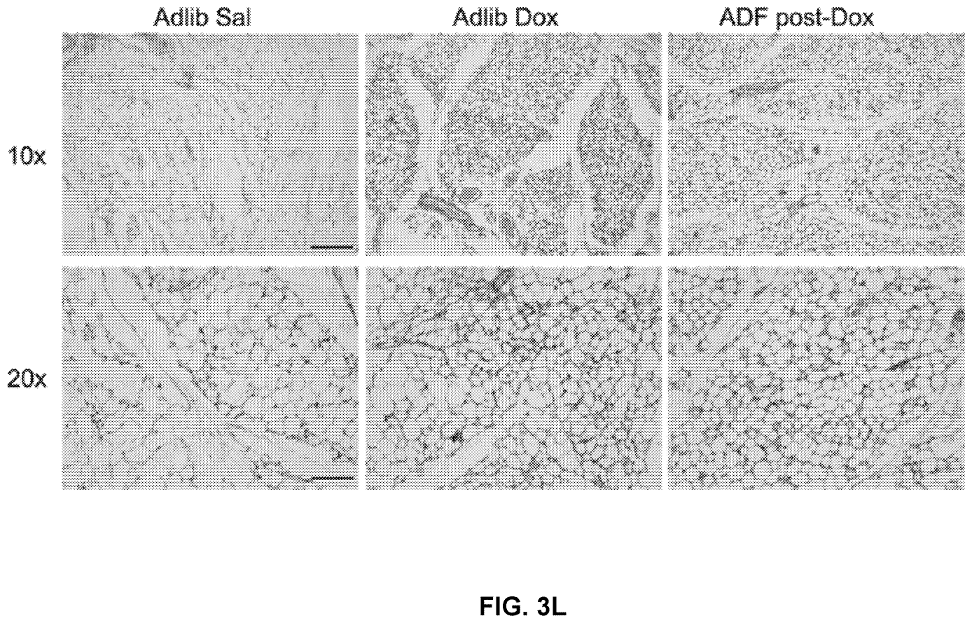

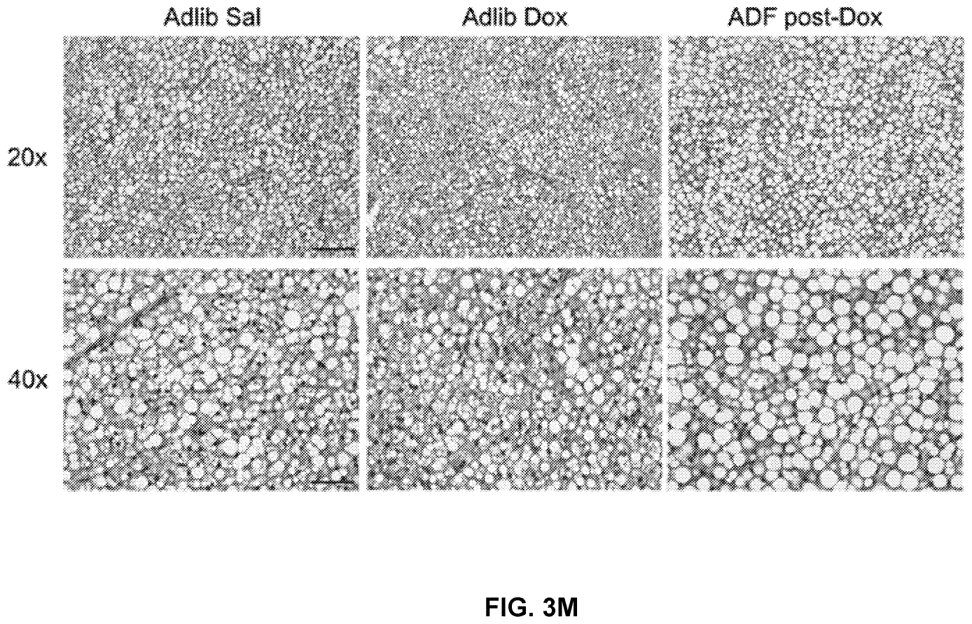

To determine how Dox+ADF increased adiposity, the mass of interscapular brown and inguinal white adipose tissue was measured. ADF induced interscapular BAT expansion while not changing inguinal white adipose tissue (iWAT) mass both in the setting of Dox (FIG. 3A, FIG. 3B, FIG. 3L, FIG. 3M) and saline (FIG. 9D, FIG. 9E) treatment, consistent with previous reports of increased BAT with ADF alone. These studies suggest that Dox+ADF caused cachexia with increasing adiposity, associated with the expansion of the interscapular brown fat depot.

Proteomics in Mice and Humans Links SLIT2, an Activator of Thermogenesis, with Dox Cardiomyopathy

Based on the morphometric observations and body composition changes in Dox+ADF mice, it was hypothesized that secreted factors might regulate both adipose tissue changes and Dox cardiotoxicity. Therefore, aptamer-based plasma proteomics analysis was utilized in mice and humans to identify a) proteins increased in Dox+ADF vs Dox mice that were b) also associated with Dox cardiomyopathy vs patients with cardiomyopathy not exposed to Dox. Among the circulating proteins in adipose-related pathways regulated in Dox+ADF mice (FIG. 3C, Table 1), SLIT2 was a significantly upregulated thermogenic protein (FIG. 3D).

TABLE 1
Adipose-related pathways

| Adipose-related pathways | Proteins |
|---|---|
| Extracellular matrix | BGN, COL18A1, COL6A1, COL9A3, DAG1, FGF2, HSPG2, IBSP, |
| organization | ITGA6, LAMC2, LRP4, MATN3, MATN4, MUSK, NCAM1, NCAN, |
| | NID2, NRXN1, NTN4, SPARC, TGFB2, TGFB3, VTN |
| Adrenergic Receptor | ATP6V1F, BDNF, CACNA2D3, COMT, CXCL8, FASLG, GNAI3, |
| Signaling Pathway | GNAS, IL13, IL17A, IL17B, IL17F, IL1A, IL1B, IL2, IL36B, IL37, |
| (Enhanced) | IL4I1, LEP, LTA, NOTUM, OSM, PLCB1, TGFB2, TGFB3, |
| | TNFSF11, TNFSF14, TNFSF15, TNFSF8 |
| Signaling by ROBO receptors | CXCL12, DAG1, DCC, NRP1, PAK5, PSMA1, PSMA3, PSMA7, |
| | PSMB1, PSMB10, PSMB2, PSMB3, PSMB4, PSMB6, PSMD10, |
| | PSMD4, PSMD7, ROBO1, SLIT2, SLIT3 |
| ROBO SLIT Signaling | ARPC5, BLK, CDH1, DCC, FLT4, HCK, JUN, LCK, MMP7, |
| Pathway | NRXN1, NRXN3, PAK5, ROBO1, SLIT2, SLIT3, TGFBR2, |
| | UNC5B, VEGFA, VEGFD |
| Adipogenesis pathway | ACVRL1, BMP4, BMP7, DDIT3, FABP4, FGF2, FGFR1, FGFR4, |
| | FGFRL1, FOXO1, HDAC8, LEP, PAXIP1, RBP1, RPS6KB1, |
| | SAP30, SIRT2, TP53, WNT5A |
| White Adipose Tissue | ANGPT2, BDNF, BMP7, CACNA2D3, CREB3L1, CREB3L2, |
| Browning Pathway | FGFR1, FGFR4, FGFRL1, GNAS, HIF1AN, LDHC, LEP, |
| | MAPK14, NPPB, PPARA, THRA, VEGFA |
| Apelin Adipocyte Signaling | APLN, GNAI3, GPX1, GPX2, GSTP1, INS, MAPK14, MAPK6, |
| Pathway | NCF2 |
| Transcriptional regulation of | ANGPTL4, FABP4, LEP, MED20, PPARA |
| white adipocyte differentiation | |

To identify circulating protein signatures associated with human Dox cardiomyopathy, the Penn Heart Failure Study (PHFS) was utilized. In PHFS, there were n=48 patients with Dox-induced cardiomyopathy and n=2249 with heart failure due to other causes. Baseline characteristics are shown in Table 2. Adjusting for covariates that differed between groups, aptamer-based proteomics was utilized in patients with available samples (n=47 vs 2167, respectively). In adjusted analysis for age, gender, race, history of diabetes mellitus, stent placement, coronary artery bypass graft surgery, organic nitrate, statin, warfarin, and insulin use, SLIT2 was the most significantly upregulated protein in Dox cardiomyopathy, both in terms of statistical significance (both alpha corrected and unadjusted p-value <1e-30) and fold change (FIG. 3E). Further, subjects in the highest tertile of SLIT2 levels exhibited significantly lower BMI and higher New York Heart Association (NYHA) classification (FIG. 3F, FIG. 3G). Other characteristics of the subjects stratified by tertiles of SLIT2 are shown in Table 3. To determine the association between SLIT2 and other circulating proteins, regression analysis were performed. In both unadjusted and adjusted (based on characteristics that differed between tertiles) analyses, SLIT2 was significantly associated with several heart failure biomarkers, such as GDF-15, NT-proBNP, and SVEP1 (Table 4). Among the biomarkers of heart failure, both adjusted and unadjusted analyses revealed that SLIT2 is most significantly associated with GDF-15 (Table 4), which promotes cardiac and skeletal muscle wasting and regulates thermogenesis. Furthermore, SLIT2 was strongly associated with its known receptors including plexin-A1, ROBO1, and ROBO2 (Table 4). These studies suggest that SLIT2 was strongly associated with Dox cardiomyopathy, advanced heart failure, and markers of cachexia (lower BMI, higher NYHA class, and increased GDF-15).

Given that SLIT2 was described as a regulator of adipose thermogenesis through UCP1, its expression was investigated in both BAT and iWAT. SLIT2 was highly upregulated both in BAT and iWAT with Dox+ADF (FIG. 3G, FIG. 3H, FIG. 3I, FIG. 3J). Altogether, these studies suggest that Dox cardiotoxicity is significantly associated with an upregulation of circulating SLIT2.

### SLIT2 is Necessary and Sufficient to Drive Doxorubicin Cardiotoxicity

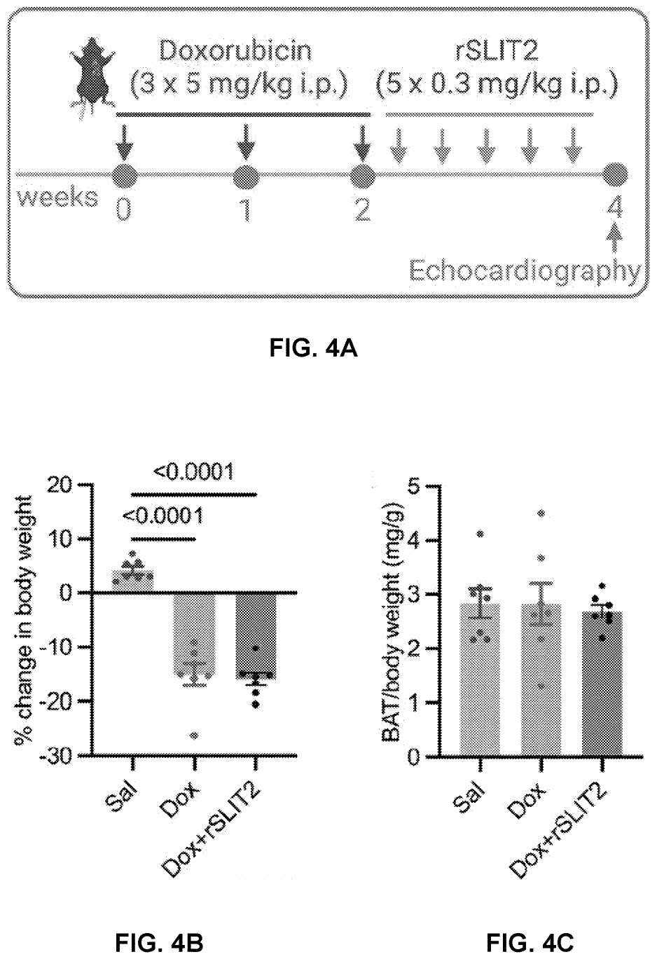

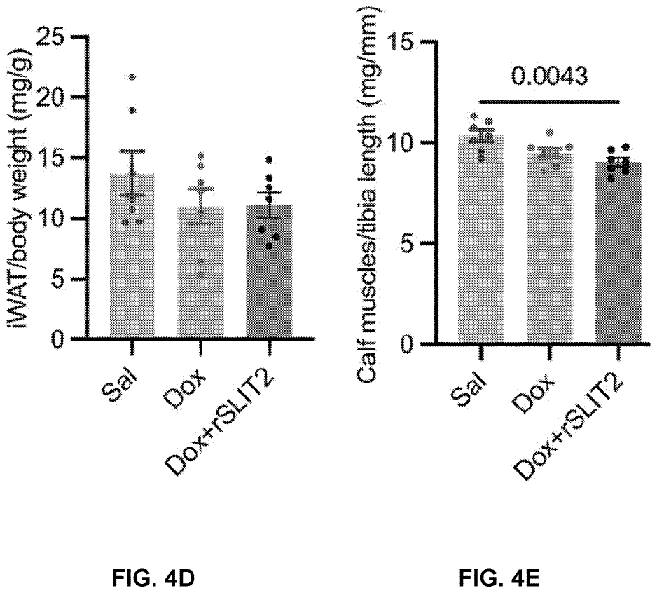

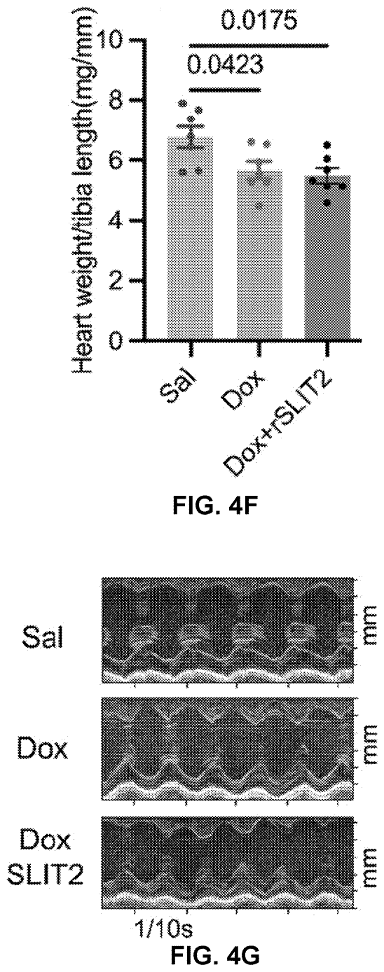

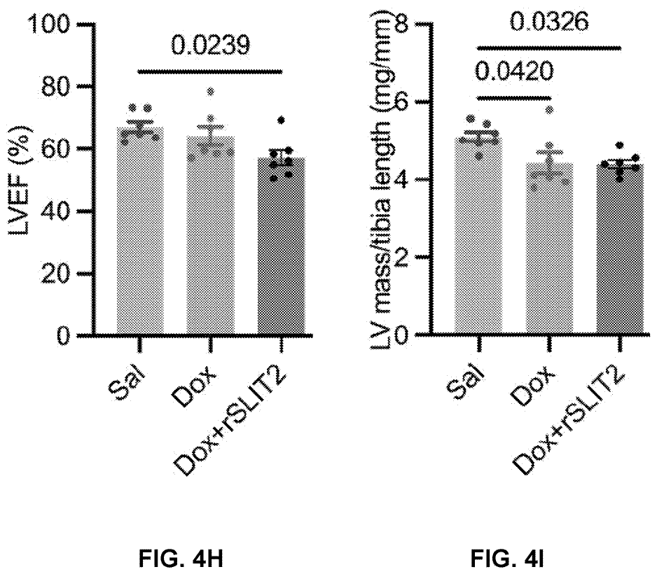

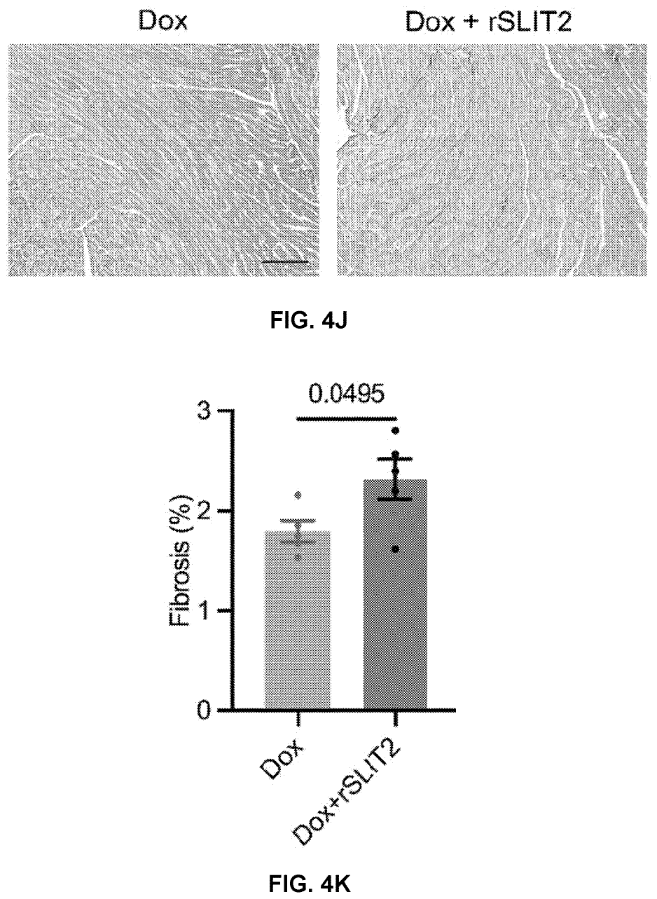

Given the significant correlations of SLIT2 and Dox cardiotoxicity, the causal role of SLIT2 in Dox-induced cardiotoxicity was further investigated. Dox (5 mg/kg intraperitoneally [i.p.] weekly for 3 weeks) was administered and followed by vehicle control vs. recombinant SLIT2 protein (rSLIT2) injections (0.3 mg/kg intraperitoneally [i.p.] for 2 weeks). Mice underwent echocardiography before euthanasia, as illustrated in FIG. 4A. There were no significant differences in body weight change and adipose tissue size between the groups (FIG. 4B, FIG. 4C, FIG. 4D), While only Dox+rSLIT2 decreased calf muscle mass indexed to tibia length compared to saline, both Dox and Dox+rSLIT2 decreased heart weight index (FIG. 4E, FIG. 4F). In this cohort, Dox alone did not change LVEF and Dox+rSLIT2 caused a significant decrease in LVEF (FIG. 4G, FIG. 4H). Both Dox and Dox+rSLIT2 had reduced LV mass/tibia length compared to saline controls (FIG. 4I). Histologically, rSLIT2 significantly increased LV fibrosis (FIG. 4J, FIG. 4K).

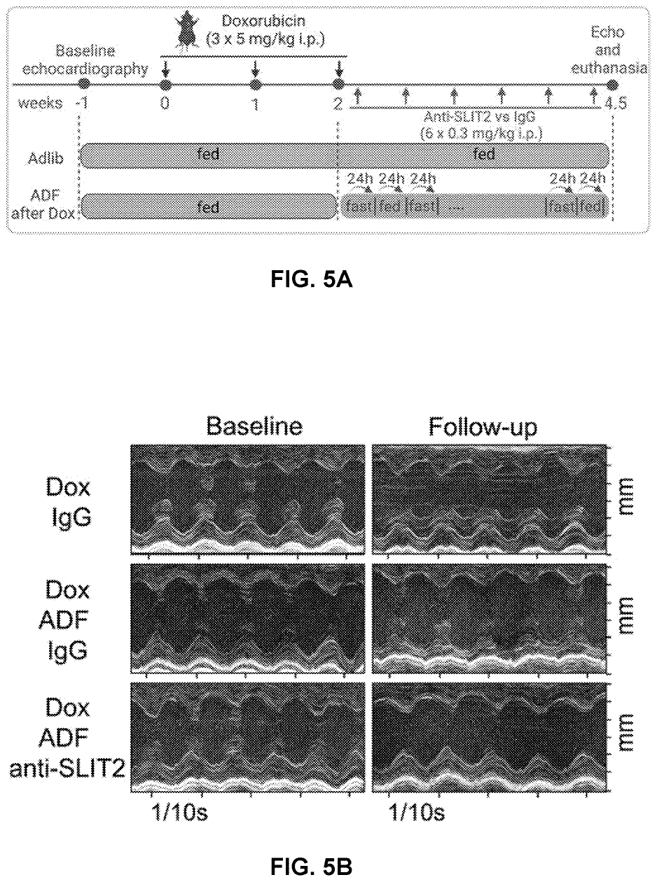

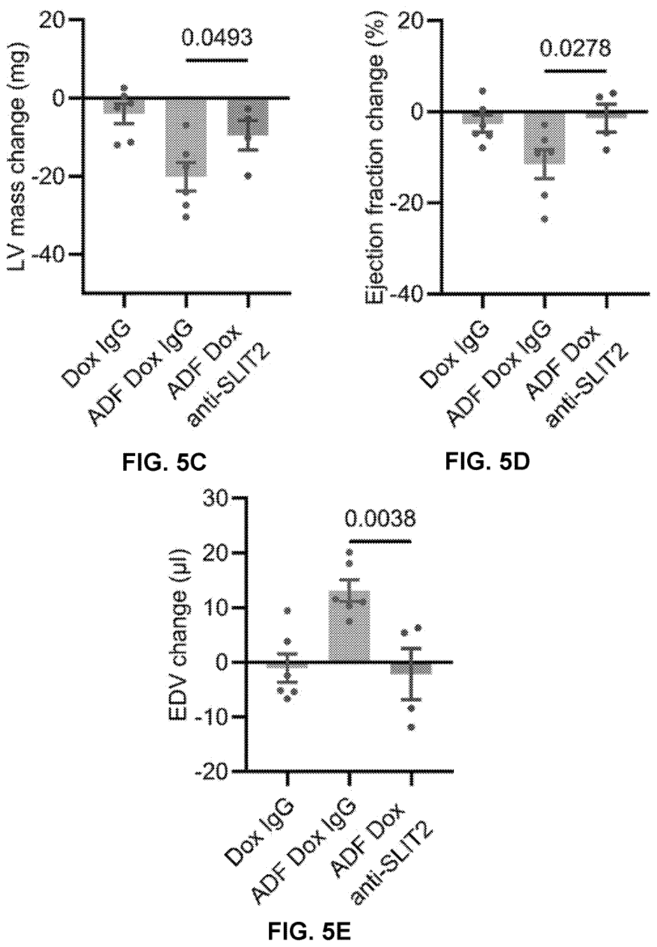

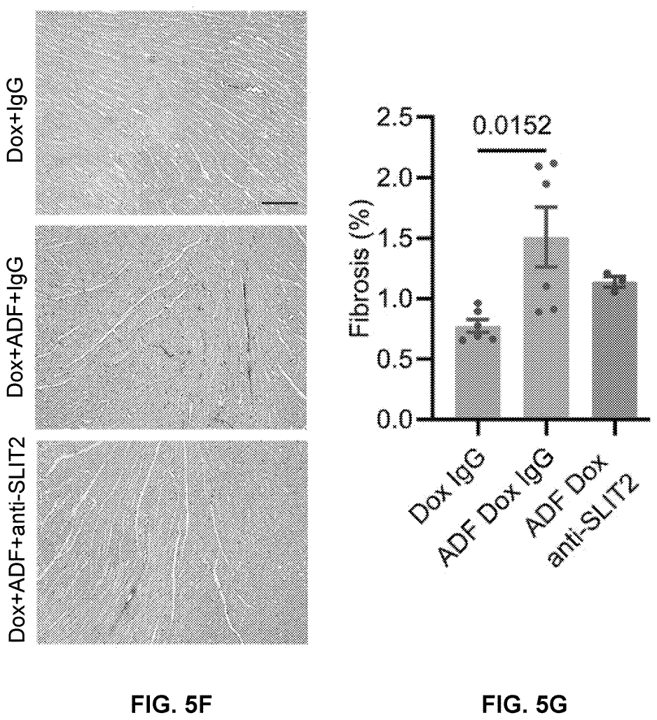

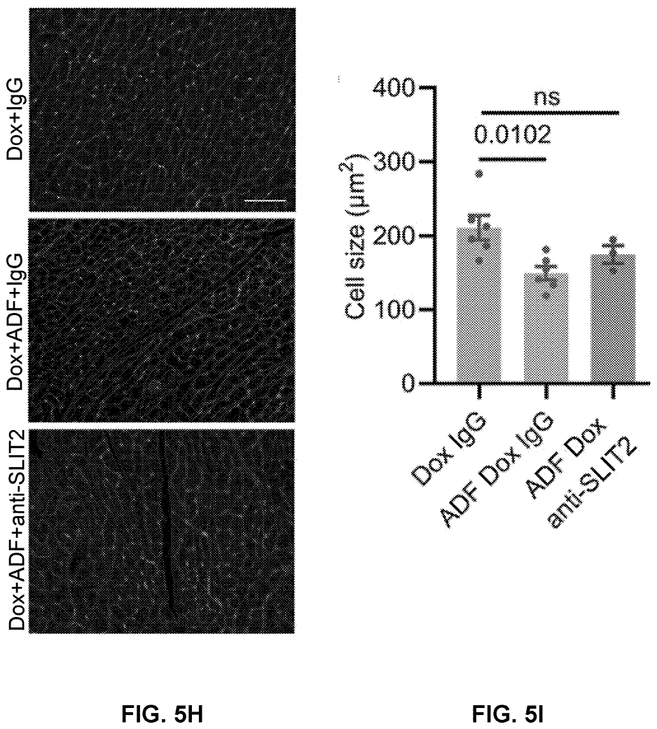

Next, it was determined if antibody-mediated neutralization of SLIT2 attenuated cardiotoxicity of Dox+ADF, particularly since SLIT2 is induced by Dox+ADF. Therefore, it was tested whether inhibition of SLIT2 with a commercial antibody targeting the N-terminal of SLIT2 (anti-SLIT2) attenuates Dox+ADF toxicity. In this experiment, mice were treated with Dox (5 mg/kg intraperitoneally [i.p.] weekly for 3 weeks) and randomized to ad libitum feeding or ADF for 2 weeks and treatment with control IgG vs. anti-SLIT2 (0.3 mg/kg intraperitoneally [i.p.] for 2 weeks), as diagramed in FIG. 5A. Echocardiography revealed that treatment with anti-SLIT2 attenuated the decreased LV mass and LVEF, as well as increased EDV induced by Dox+ADF (FIG. 5B, FIG. 5C, FIG. 5D, FIG. 5E). Histologically, anti-SLIT2 treatment attenuated the increased fibrosis and reduced cardiomyocyte cross-sectional area induced by Dox+ADF (FIG. 5F, FIG. 5G, FIG. 5H, FIG. 5I). Altogether, these findings suggest a causal role of SLIT2 in doxorubicin cardiotoxicity.

Alternate-Day Fasting after Doxorubicin Activates SLIT2-Target UCP1, which is Necessary for Doxorubicin Cardiotoxicity

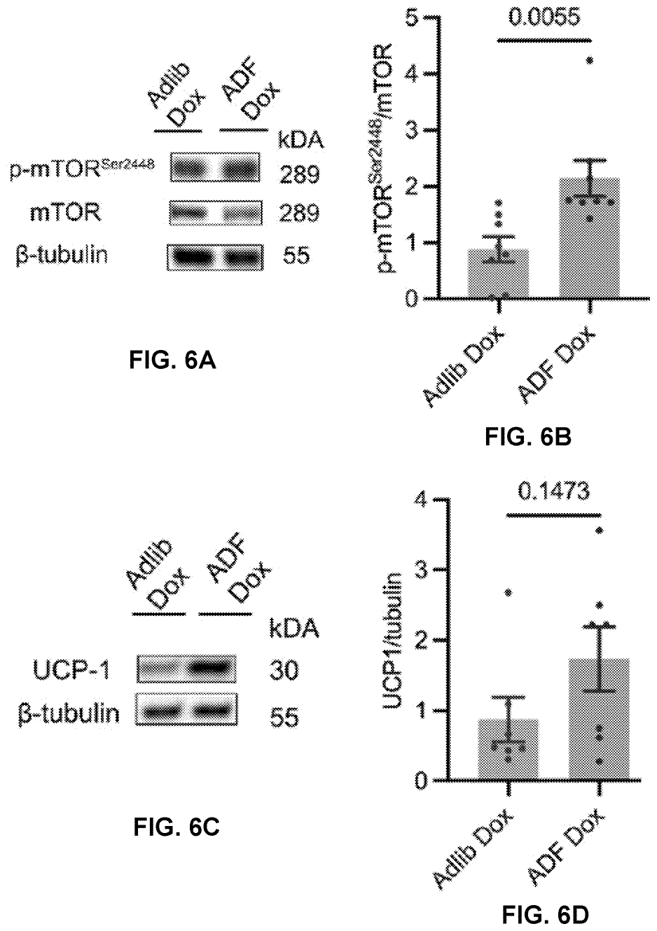

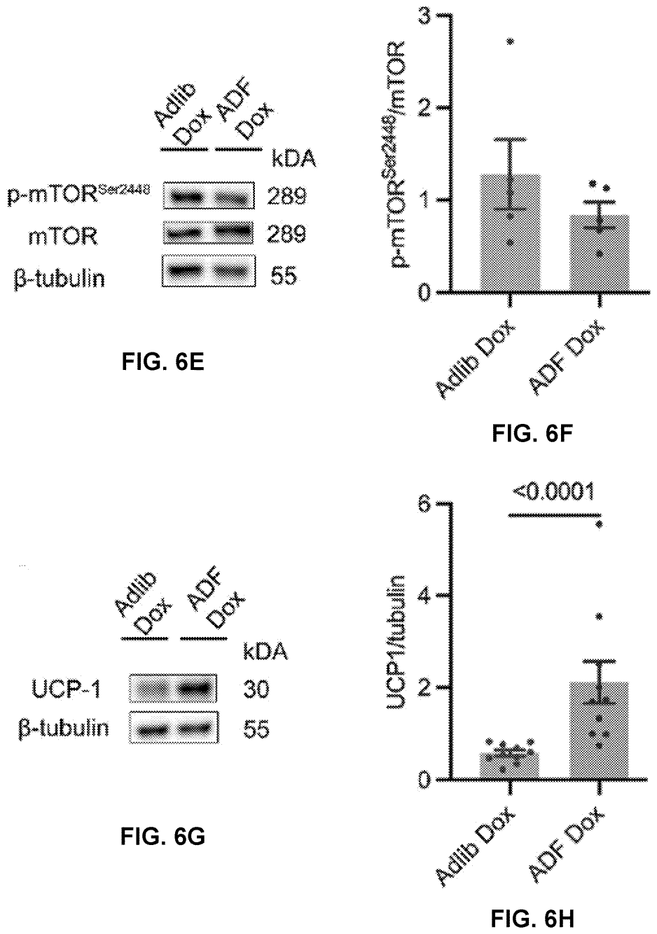

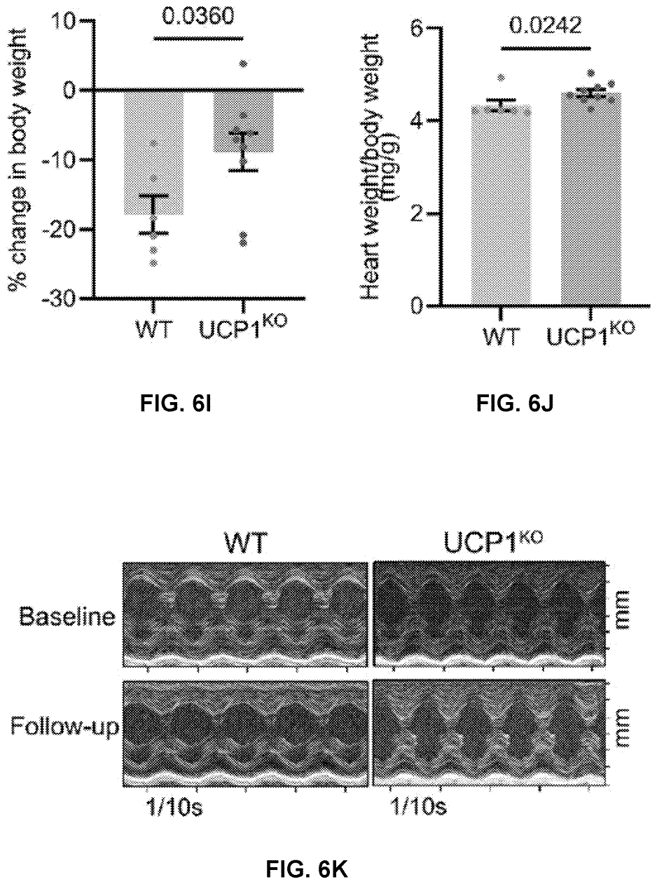

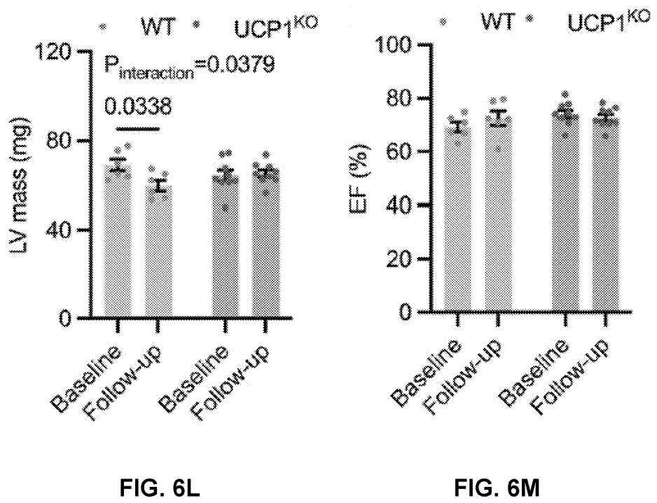

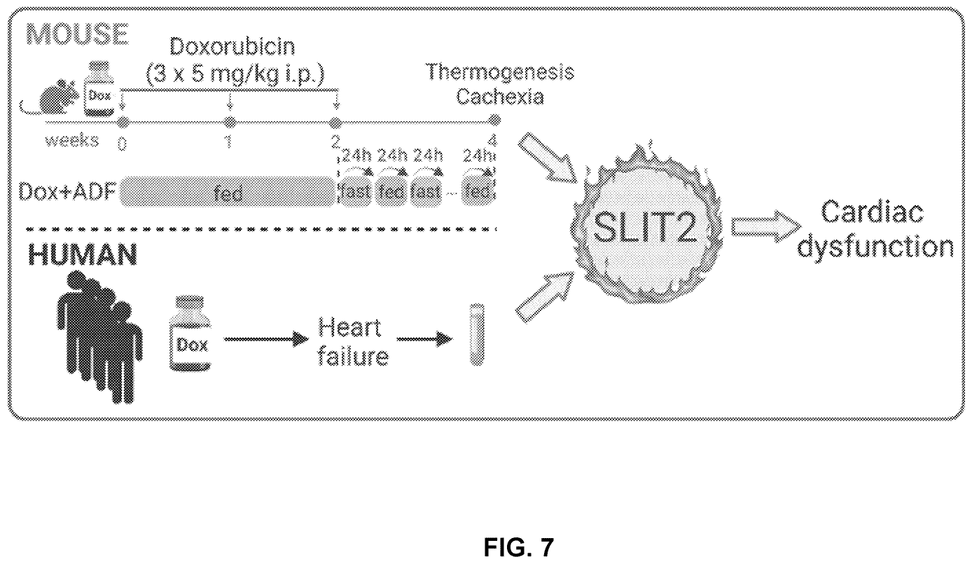

To further examine the role of adipose tissue thermogenesis, the molecular changes in adipose tissue were explored. Dox+ADF induced mammalian target of rapamycin (mTOR) phosphorylation at Ser2448 (FIG. 6A, FIG. 6B), which regulates adipose growth and thermogenesis, in BAT. However, ADF did not significantly alter UCP1 levels in BAT compared with Dox treatment alone (FIG. 6C, FIG. 6D). In contrast, Dox+ADF did not change mTOR phosphorylation but caused significant upregulation of UCP1 levels in iWAT compared to Dox alone (FIG. 6E, FIG. 6F, FIG. 6G, FIG. 6H).

Since UCP1 was significantly increased in iWAT in the Dox+ADF group, it was hypothesized that UCP1 itself is necessary for Dox cardiotoxicity. To test this hypothesis, UCP1 whole-body knockout (UCP1^{KO}) mice were utilized. UCP1^{KO} and littermate control mice were treated with Dox (5 mg/kg intraperitoneally [i.p.] weekly for 3 weeks) and underwent echocardiography both before and after Dox treatment. Since the wild-type mice started losing more than 20% of their initial body weight earlier than expected, these mice were urgently euthanized 5 days after the last dose of Dox. UCP1^{KO} mice exhibited less severe body weight loss and increased heart weight normalized to body weight (FIG. 6I, FIG. 6J). Echocardiography showed that UCP1^{KO} attenuated Dox-induced LV mass loss at euthanasia vs. baseline compared with wildtype littermate controls and there were no changes in LVEF in both groups after Dox treatment (FIG. 6K, FIG. 6L, FIG. 6M, FIG. 6N). These results support that the deletion of UCP1 attenuates Dox-mediated cardiac atrophy.

### Discussion

In this study, a combination of human plasma and murine models were used to demonstrate that SLIT2 drives Dox cardiotoxicity, in part through brown adipose thermogenesis. The main conclusions of this study are that 1) Dox followed by ADF leads to enhanced Dox-induced cachexia and cardiotoxicity; 2) Dox followed by ADF increases adiposity, brown adipose mass, and SLIT2; 3) SLIT2 is necessary and sufficient to drive Dox cardiotoxicity in mice; 4) SLIT2 is the most significantly upregulated circulating protein in human Dox cardiomyopathy in adjusted analysis and correlates with markers of advanced heart failure/cachexia; 5) UCP1 deletion also attenuates Dox cardiotoxicity. Altogether, these studies suggest that SLIT2 and its target UCP1 accelerate Dox cardiotoxicity, and that SLIT2 may be a therapeutic target in Dox cardiomyopathy. This study also highlights that extreme forms of IF like ADF may not be advisable after Dox chemotherapy.

A prior study showed that six weeks of ADF was not tolerated when initiated before Dox and continued throughout the treatment course. In the present study, a shorter intervention was employed which began after Dox completion, in part to avoid potential mortality, but also to prevent potential confounding of fasting before and during Dox treatment in the prior model. Although Dox+ADF in the current study was also toxic, it was mechanistically distinct from the prior ADF model. These differences might be due to the timing of ADF or potentially to the duration of ADF. Because fasting is a complex physiological intervention, different types of IF may demonstrate diverse results even within the same pathological setting. Consequently, future studies should explore alternative IF strategies and/or dietary interventions for the treatment or prevention of Dox cardiotoxicity, including time-restricted eating or ADF at various doses and timepoints with respect to Dox administration. However, from a translational point-of-view, the current and prior studies provide cautionary evidence against the use of ADF after Dox administration. Finally, the identification of SLIT2 as a target highlights the translational applicability of the ADF model, although it remains uncertain what the stimulus for SLIT2 is in patients with Dox cardiomyopathy.

Mechanistically, previous studies have demonstrated that 2 weeks of ADF alone in mice induce UCP1-mediated thermogenesis only in iWAT, with no significant effects observed in BAT. Similarly, increased expression of SLIT2 was shown to activate UCP1-induced thermogenesis specifically in iWAT. These findings are consistent with these observations, as Dox+ADF induces SLIT2 expression in both iWAT and BAT, yet UCP1 upregulation occurs only in iWAT. The discrepancy in UCP1 upregulation between brown and beige adipocytes may be attributed to a previously proposed β-adrenergic receptor-independent thermogenesis pathway unique to iWAT. This pathway could potentially include SLIT2-induced thermogenesis, which appears to be unaffected by adrenergic inhibition. Additionally, a proteomics study of adipose depots revealed that while UCP1 levels increase in iWAT after ADF, they remain substantially lower (by 50-fold) compared with the BAT depot, suggesting a less prominent role for UCP1-induced thermogenesis in iWAT, although this needs further investigation. Furthermore, it is plausible that SLIT2 may activate UCP1-independent thermogenesis pathways in both BAT and iWAT.

In addition to its role in activating thermogenesis, SLIT2 may exert direct effects on cardiac tissue through the roundabout guidance receptors (ROBO1-4). Notably, a recent genome-wide association study has identified ROBO2 as a susceptibility gene for Dox-induced cardiomyopathy. Moreover, SLIT2-ROBO1 signaling pathway has been implicated in promoting cardiac fibrosis, a hallmark of Dox cardiotoxicity. The increased myocardial fibrosis observed in mice that underwent Dox+ADF may be attributed to the activation of SLIT-ROBO signaling in heart, as supported by these findings of increased fibrosis in myocardium from mice treated with Dox+rSLIT2.

Taken together, these findings suggest that the SLIT2 pathway holds promise for the treatment or prevention of Dox cardiomyopathy and possibly other forms of heart failure. Moving forward, further exploration into the identification and functions of SLIT2 receptors, as well as the roles of SLIT2 and its fragments, would allow elucidation of additional aspects of SLIT2 signaling and potential targets to mitigate Dox cardiotoxicity. These discoveries have potentially important translational implications and suggest increased clinical vigilance of the relationship between SLIT2 and doxorubicin-associated cardiotoxicity.

TABLE 2
General characteristics of PHFS subjects with doxorubicin-
induced cardiomyopathy (CMP) vs other cardiomyopathy.

| Clinical variable | Doxorubicin | Other | |
|---|---|---|---|
| Mean ± SEM or n (%) | CMP (n = 48) | CMP (n = 2249) | p value |
| Age (years) | 54.5 (50.5, 58.5) | 56.8 (56.2, 57.4) | 0.263 |
| Male sex | 18 (37.5%) | 1498 (66.6%) | <0.0001 |
| Race/Ethnicity | 10 (20.8%) | 459 (20.4%) | >0.99 |
| African American | | | |
| BMI (kg/m2) | 28.25 (26.2, 30.4) | 30.24 (29.9, 30.5) | 0.066 |
| Systolic BP (mmHg) | 111 (105, 117) | 114.9 (114, 116) | 0.208 |
| Diastolic BP (mmHg) | 69.6 (66.2, 72.9) | 69.6 (69.2, 71) | 0.962 |
| Diabetes mellitus | 7 (14.58%) | 652 (28.99%) | 0.034 |
| Atrial fibrillation or flutter | 12 (25%) | 824 (36.64%) | 0.128 |
| LV ejection fraction (%) | 38 (33.2, 42.8) | 33.2 (32.5, 33.9) | 0.053 |
| History of stent | 4 (8.3%) | 506 (22.5%) | 0.021 |
| History of CABG | 428 (2.17%) | 58 (4.01%) | >0.99 |
| eGFR | 60.4 (52.6, 68.3) | 57.2 (56.1, 58.3) | 0.42 |
| NYHA Class | | | ns |
| NYHA 1 | 11 (21.7%) | 405 (17.5%) | |
| NYHA 2 | 23 (47.9%) | 999 (44.7%) | |
| NYHA 3-4 | 14 (30.4%) | 845 (37.8%) | |

Medication Use

| | | | |
|---|---|---|---|
| Beta Blocker | 38 (79.2%) | 1986 (88.3%) | 0.067 |
| Aspirin | 23 (47.9%) | 1300 (57.8%) | 0.185 |
| ACEI/ARBs | 37 (77.1%) | 1910 (84.9%) | 0.153 |
| Hydralazine | 1 (2.1%) | 192 (8.5%) | 0.181 |
| Nitrates | 1 (2.1%) | 360 (16%) | 0.004 |
| Statin | 14 (29.2%) | 1188 (52.8%) | 0.001 |
| CCBs | 1 (2.1%) | 213 (9.5%) | 0.125 |
| Warfarin | 10 (20.8%) | 852 (37.9%) | 0.016 |
| Insulin | 1 (2.1%) | 294 (13.1%) | 0.016 |
| Current smoking | 1 (2.1%) | 204 (9.07%) | 0.121 |

BMI (Body mass index), BP (Blood pressure), LV (Left ventricular), PCI (Percutaneous coronary intervention), CABG (Coronary artery bypass graft), eGFR (Estimated glomerular filtration rate), NYHA (New York Heart Association class), ACEI/ARBs (Angiotensin-converting enzyme inhibitors/angiotensin receptor blockers), CCBs (Calcium channel blockers).

TABLE 3
General characteristics of the overall cohort from
PHFS, across all 3 tertiles of plasma SLIT2.

| Clinical variable | Lowest tertile | Middle tertile | Highest tertile | |
|---|---|---|---|---|
| Mean ± SEM or n (%) | (n = 743) | (n = 747) | (n = 745) | p value |
| Plasma SLIT2 levels | 1063.4-4770.8 | 4770.8-6024.1 | 6024.1-181087.4 | |
| Age (years) | 50.3 (49.3, 51.3) | 53.4 (52.3, 54.5) | 60.4 (59.1, 61.6) | <0.0001 |
| Male sex | 460 (61.9%) | 510 (68.3%) | 513 (68.9%) | 0.0078 |
| Race/Ethnicity | 190 (25.4%) | 165 (22%) | 116 (15.5%) | <0.0001 |
| African American | | | | |
| BMI (kg/m2) | 31.5 (30.8, 31.9) | 29.2 (28.7, 29.7) | 27.7 (27.3, 28.2) | <0.0001 |
| Systolic BP (mmHg) | 114.6 (113, 116) | 113.9 (112, 115) | 110.7 (109, 112) | 0.0004 |
| Diastolic BP (mmHg) | 70.4 (69.6, 71.3) | 69.7 (68.8, 70.5) | 66.1 (65.3, 66.9) | <0.0001 |
| Diabetes mellitus | 156 (20.8%) | 214 (28.5%) | 272 (36.3%) | <0.0001 |
| Atrial fibrillation | 203 (27.1%) | 275 (36.7%) | 343 (45.8%) | <0.0001 |
| or flutter | | | | |
| History of stent | 130 (17.4%) | 156 (20.8%) | 212 (28.3%) | <0.0001 |
| History of CABG | 89 (11.9%) | 133 (17.7%) | 195 (26%) | <0.0001 |
| eGFR | 56.2 (54.2, 58.1) | 54.9 (53, 56.8) | 46.6 (45, 48.2) | <0.0001 |
| NYHA Class | 146 (19.8%) | 170 (22.9%) | 68 (9.2%) | <0.0001 |
| NYHA 1 | 373 (50.6%) | 324 (43.6%) | 295 (39.9%) | |
| NYHA 2 | 218 (29.6%) | 249 (33.5%) | 376 (50.9%) | |
| NYHA 3-4 | | | | |

Medication Use

| | | | | |
|---|---|---|---|---|
| Beta Blocker | 655 (87.5%) | 678 (90.4%) | 640 (85.5%) | 0.0122 |
| Aspirin | 402 (53.7%) | 429 (57.2%) | 456 (60.9%) | 0.0183 |
| ACEI/ARBs | 650 (86.7%) | 646 (86.1%) | 607 (81%) | 0.0044 |
| Hydralazine | 58 (7.7%) | 66 (8.8%) | 67 (9%) | 0.6634 |
| Nitrates | 106 (14.2%) | 120 (16%) | 131 (17.5%) | 0.2098 |
| Statin | 366 (48.9%) | 391 (52.1%) | 412 (55%) | 0.0598 |
| CCBs | 80 (10.7%) | 60 (8%) | 65 (8.7%) | 0.181 |
| Warfarin | 236 (31.5%) | 271 (36.1%) | 344 (45.9%) | <0.0001 |
| Insulin | 70 (9.4%) | 93 (12.4%) | 121 (16.2%) | 0.0004 |
| Current smoking | 96 (12.8%) | 58 (7.7%) | 49 (6.5%) | 0.0001 |

BMI (Body mass index), BP (Blood pressure), LV (Left ventricular), PCI (Percutaneous coronary intervention), CABG (Coronary artery bypass graft), eGFR (Estimated glomerular filtration rate), NYHA (New York Heart Association class), ACEI/ARBs (Angiotensin-converting enzyme inhibitors/angiotensin receptor blockers), CCBs (Calcium channel blockers).

TABLE 4
Circulating proteins associated with SLIT2.

| Unadjusted | | Univariable regression analysis | |
|---|---|---|---|
| Protein | Std Estimate | p-value | FDR-corrected p-value |

Biomarkers of heart failure

| | | | |
|---|---|---|---|
| GDF-15 | 0.415 | 6.5E−153 | 4.4E−150 |
| SVEP1 | 0.388 | 9.1E−138 | 4.9E−135 |
| NT-proBNP | 0.372 | 2.2E−122 | 9.3E−120 |
| SVEP1 | 0.386 | 2.7E−136 | 1.4E−133 |
| NT-proBNP | 0.338 | 5.1E−98 | 1.3E−95 |
| Adiponectin | 0.351 | 1.0E−107 | 3.3E−105 |
| ST2 | 0.244 | 2.2E−48 | 1.7E−46 |

Receptors of SLIT2

| ROBO1 | 0.482 | 1.9E−194 | 3.8E−191 |
|---|---|---|---|
| PLXNA1 | 0.556 | 1.7E−275 | 1.4E−271 |
| ROBO2 | 0.285 | 5.8E−69 | 7.7E−67 |
| Adjusted | | Multivariable regression analysis | |
| Protein | Std Estimate | p-value | FDR-corrected p-value |

Biomarkers of heart failure

| | | | |
|---|---|---|---|
| GDF-15 | 0.441 | 3.4E−89 | 1.5E−86 |
| SVEP1 | 0.307 | 9.3E−57 | 1.5E−54 |
| NT-proBNP | 0.301 | 2.7E−52 | 3.9E−50 |
| SVEP1 | 0.311 | 5.2E−59 | 9.1E−57 |
| NT-proBNP | 0.255 | 2.5E−39 | 2.2E−37 |
| Adiponectin | 0.268 | 3.3E−46 | 3.7E−44 |
| ST2 | 0.145 | 9.1E−15 | 2.6E−13 |

Receptors of SLIT2

| | | | |
|---|---|---|---|
| ROBO1 | 0.492 | 1.5E−201 | 8.8E−198 |
| PLXNA1 | 0.498 | 1.3E−197 | 6.8E−194 |
| ROBO2 | 0.295 | 4.1E−66 | 8.8E−64 |

Adjustments were made based on the clinical characteristics that were significantly different across all the tertiles of SLIT2 shown in Table S3. Duplicate measurements from some proteins indicate results from different aptamers targeting the same protein.

### Materials and Methods

### Human Studies

To identify the proteins associated with doxorubicin cardiomyopathy, plasma samples from subjects who had a history of doxorubicin-induced cardiomyopathy (n=48) vs. subjects with cardiomyopathy not exposed to doxorubicin (n=2249) in the PHFS, a prospective cohort study of patients with HF recruited at the University of Pennsylvania (Philadelphia, PA), Case Western Reserve University (Cleveland, OH), and the University of Wisconsin (Madison, WI) between 2003 and 2011. Patients with a clinical diagnosis of HF as determined by an HF specialist were enrolled. At the time of enrollment, standardized questionnaires were administered to participants and their physicians to obtain detailed clinical data. Participants with expected mortality of ≤6 months from a noncardiac condition, as judged by their treating physician, mechanical circulatory support, or inability to provide informed consent, were excluded. Participants provided written informed consent, and the study was approved by an institutional review board from each of the participating centers.

### Plasma Protein Quantification

Human and murine plasma samples were analyzed using the SomaScan® assay. SomaScan® is a multiplexed, modified aptamer-based binding assay. The SomaScan® assay utilizes Slow-Off-rate Modified Aptamer reagents, which are chemically modified nucleotides, to bind and quantify target proteins in relative fluorescent units directly proportional to the amount of target protein in the sample.

### Pathway Analysis

Pathway analysis for murine plasma proteomics was performed using Ingenuity Pathway Analysis software (Qiagen; Hilden, Germany). Proteins were identified according to their UniProt identifier annotation. The totality of proteins included in the SomaScan assay was used as the reference set and both direct and indirect experimentally confirmed relationships from all species were included. The Core analysis module in Ingenuity Pathway Analysis was used to perform pathway analysis on the differentially expressed proteins. This analysis identifies specific canonical pathways in which the changes are induced by Dox+ADF in mice. The analysis calculates a P-value (Fisher exact test and right tailed), quantifying the overlap, and a Z score, quantifying the likelihood and direction (up or downregulated), between the plasma proteomics pattern and known canonical pathways.

### Rodent Studies

Wild-type male mice of the C57BL/6 strain and male and female UCP1^{KO} mice (#003124) were obtained from Jackson Laboratories. Mice were housed in groups of up to n=2-5 mice/cage and fed standard chow (Lab Diet, 5053; providing 3.4 Kcal/g with 62.1% Kcal derived from carbohydrates, 13.2% from fats, and 24.6% from protein) on a 6:00 PM to 6:00 AM dark-light cycle, in a temperature controlled specific pathogen-free facility. Alternate day fasting (ADF) was performed with total food deprivation and ad libitum access to water while mice were housed on aspen shavings-bedding from 11:00 AM to 11:00 AM of the following day to implement alternate periods of 24 h fasting and feeding, with change in bedding. Non-fasted control mice were simultaneously provided fresh food with a change in aspen shavings-bedding. Terminal studies on mice were initiated between 10:00 AM and 12:00 PM after an overnight period of feeding (i.e., on a fed day).

### Reagents

Pharmacologic-grade doxorubicin hydrochloride (50 mg) was purchased from United States Pharmacopeia (Cat #1225703, USP, R11760, Rockville, MD, USA) and dissolved in 20 ml of molecular grade water to get a 2.5 mg/ml stock, stored at −80 °C, was used in all animal experiments with Dox.

Recombinant human SLIT2 (rSLIT2, 50 μg) containing a C-terminal 6-His tag was purchased from Biotechne/R&D Systems (Cat #8616-SL-050, Minneapolis, MN, USA). Lyophilized protein was reconstituted in 200 μl of molecular biology grade water to get 250 μg/ml stock, stored at 4 °C upon reconstitution and used in all animal experiments with rSLIT2.

Anti-human/mouse SLIT2 antibody (anti-SLIT2, 100 μg) was purchased from Biotechne/R&D Systems (Cat #MAB5444, Minneapolis, MN, USA) and reconstituted in 200 μl of sterile PBS to get 500 μg/ml stock, stored at 4 °C upon reconstitution and used in all animal experiments with anti-SLIT2.

### Doxorubicin Cardiotoxicity Studies

In age and sex-matched 8-9 weeks-old adult mice, cardiotoxicity was induced by intraperitoneal (i.p.) injection of Dox at specified doses. In ADF experiments, mice were treated with to Dox 5 mg/kg once weekly×3 weeks, at the same time each day, followed by ad libitum diet vs. ADF for 2 weeks. Echocardiography were performed and read blindly by a cardiologist. LV mass and EF were obtained using the Vevostrain package in Vevo (Fujifilm Visualsonics, Toronto, Canada). Volumetric analyses were performed using the VevoStrain speckle-tracking software included in the VevoLab workstation (Fujifilm Visualsonics, Toronto, ON, USA).

### Recombinant Human SLIT2 Experiment

C57BL/6 mice were randomized to saline vs. Dox (5 mg/kg i.p. weekly, 3 weeks, n=7/group) and Dox-treated mice were randomized to recombinant SLIT2 (0.3 mg/kg×6) vs vehicle control (molecular biology grade water).

### Human/Mouse SLIT2 Antibody Experiment

C57BL/6 mice were treated with Dox (5 mg/kg i.p. weekly, 3 weeks, n=6/group) and randomized to adlib IgG, ADF after Dox+IgG, and ADF after Dox+anti-SLIT2 antibody injections (0.3 mg/kg i.p.×6) vs vehicle control (sterile PBS).

### Assessment of Body Weight and Body Composition

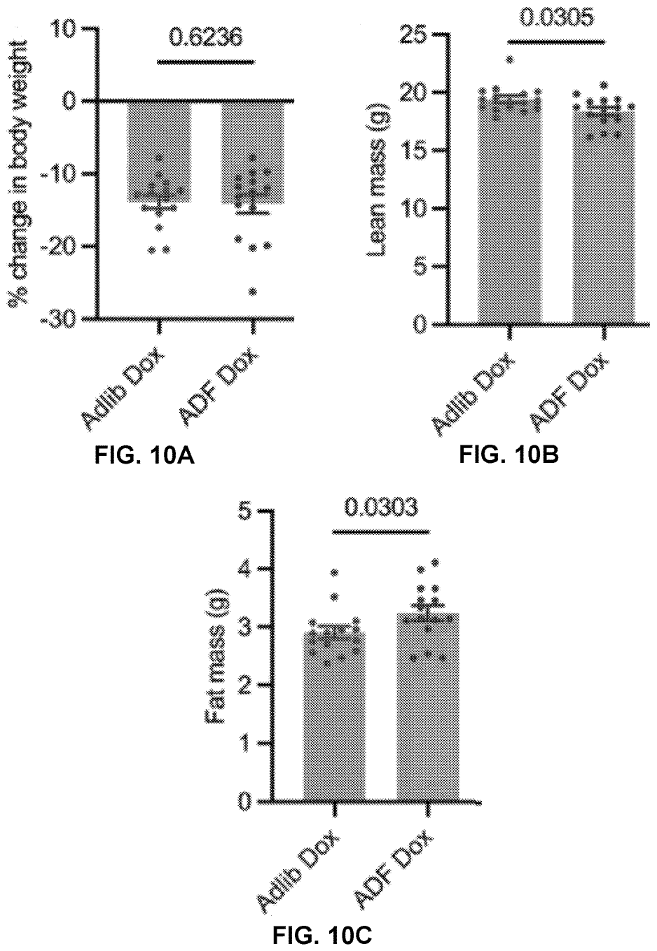

Body weight of individual animals was measured intermittently until the end of the experimental protocol. Body composition (fat mass and lean mass) in live animals was analyzed by a rodent EchoMRI. EchoMRI-based body composition analysis before euthanasia revealed Dox+ADF led to a reduction in lean mass and an unexpected increase in fat mass compared to the Dox-alone group (n=15/group) (FIG. 10A, FIG. 10B, FIG. 10C). To validate the observations of increased fat mass in the ADF group and to expand the investigation, paired body composition analysis was conducted in a new set of mice (n=10/group) at baseline before the 1^{st} dose of Dox and at the time of euthanasia (FIG. 2A, FIG. 2B, FIG. 2C, FIG. 2D).

### Histologic Analyses

Histological analyses were performed. Briefly, mouse heart tissues were fixed immediately after euthanasia in 4% formalin at room temperature overnight, then stored in phosphate-buffered saline (PBS) at 4 °C and embedded in paraffin wax. Paraffin sections were cut and mounted on glass slides. Images of Picrosirius red (PSR)/Fast green-stained tissue slices of paraffin-embedded murine cardiac tissue were acquired with an Olympus BX43F light microscope. 20× images of PSR/Fast green-stained tissue slices were then quantified using the ImageJ software (National Institutes of Health). High-resolution images of WGA-stained tissue slices of paraffin-embedded murine cardiac tissue were acquired using the 20× objective of a Zeiss LSM 900 confocal microscope. Cell size was measured by a blinded observer using the ImageJ software (National Institutes of Health).

### Total Protein Extraction

Freshly isolated mouse heart samples were snap frozen in liquid nitrogen after euthanasia and stored at −80 °C. Tissues were homogenized in ice-cold lysis buffer (composition: 50 mM Tris HCl [pH 7.4], 2.5 mM EDTA, 10 mM EGTA, 20 mM NaF, 25 mM Na4P2O7, 2 mM Na3VO4, 25 mM NaCl, and 0.2% NP-40) containing protease and phosphatase inhibitor cocktail (#PI78442; Thermo Scientific) and then centrifuged at 12000 g for 20 min at 4 °C. After discarding the pellets, supernatants (heart lysates) were stored at −80 °C.

Similar to hearts, adipose tissues were homogenized in the ice-cold lysis buffer described above. After centrifuging at 18000 g for 20 min at 4 °C, supernatants were taken out to a new tube with the pipette tips pointing at mid-level inside the lysate without contamination by the upper-fat cake layer. This process was repeated after supernatants were centrifuged again at 18000 g for 10 min at 4 °C. After discarding the pellet, new supernatants (adipose lysates) were stored at −80 °C.

### Western Blot Analysis

Western blots were performed on mice. Briefly, equal amounts of total protein (20 μg for total protein) were separated by 4-20% SDS-PAGE and transferred onto polyvinylidene fluoride membranes (Millipore Sigma #IPVH00010). The membranes were blocked with 5% nonfat milk at room temperature for 1 h and incubated overnight at 4 °C with the primary antibodies, and the membranes were washed and further incubated with secondary antibodies at room temperature for 1 h. The immune complexes were visualized by enhanced chemiluminescent methods, and the band intensity was quantified using Image J software (National Institutes of Health). Primary antibodies employed were as follows: phospho-mTOR (Ser2448) (Cell Signaling, #5536s, diluted 1:1000), mTOR (Cell Signaling, #2972s, diluted 1:1000), β-tubulin (Cell Signaling, #2146s, diluted 1:1000), SLIT2 (Abcam, #ab134166, diluted 1:1000; CST, #47600, diluted 1:1000), UCP1 (Abcam, #ab10983, diluted 1:1000; Cell Signaling, #14670, diluted 1:1000), and vinculin (Cell Signaling, #13901 1:1000, Secondary antibodies employed were as follows: anti-mouse IgG (Cell Signaling, #7076, diluted 1:2000), anti-rabbit IgG (Cell Signaling, #7074, diluted 1:2000).

### Quantification and Statistical Analysis

When normality assumptions were confirmed, populations were compared using two-tailed Student's t-test for two groups and one-way analysis of variance (ANOVA) for three groups, or two-way ANOVA for comparisons of multiple parameters. Normality was visualized by Q-Q plot and confirmed by Shapiro-Wilk test. Overall ANOVA F-statistics were significant for all presented comparisons, and multiple comparisons were corrected using Sidak's test. When normality assumptions were rejected, non-parametric Mann-Whitney or Kruskal-Wallis tests were used. Specific tests used to analyze each experiment are described in the figure legends. Data are presented as mean±SEM unless indicated. Two-sided P values less than 0.05 were considered to be statistically significant. Statistical analyses were performed in GraphPad Prism v10.1.2 (La Jolla, CA). Statistical analyses for human plasma proteomics were performed using the MATLAB statistics and machine learning toolbox (Matlab 2016b, the Mathworks; Natwick, MA).

### Example 2: Alternate Day Fasting (ADF) Potentiates Dox Cardiotoxicity and Increases Brown Adipose Mass

To evaluate the effect of short-term fasting after doxorubicin (Dox) treatment and the associated changes in cardiotoxicity, the following experiments were performed.

Chow-fed C57BL/6J mice were randomized to ad-lib feeding or alternate day fasting after Dox treatment (5 mg/kg i.p.×3 doses), denoted Adlib Dox and ADF post-Dox respectively, followed by body composition analysis before euthanasia. Measurements of lean mass and body fat content were obtained by EchoMRI. ADF post-Dox mice displayed decreased lean mass, increased fat mass, decreased heart weight to tibia length ratio, decreased calf muscles to body weight ratio, decreased tibialis anterior to body weight ratio, and increased liver weight to body weight ratio. The ADF post-Dox mice further exhibited exacerbated cardiac and skeletal muscle mass loss and increased fat mass.

Additionally, the ADF post-Dox treatment potentiated Dox cardiotoxicity. Echocardiographic data (under 2% Avertin, IP) in C57BL/6J mice randomized to ad-lib feeding or ADF after Dox and treated with Dox (5 mg/kg i.p.×3 doses). Echocardiography was performed on a feeding day. Mice undergoing ADF after Dox experienced decreased left ventricular ejection fraction, decreased left ventricular mass, and increased EVD. The ADF after Dox-treated mice also had an increased ratio of brown adipose tissue compared to their body weight. These results demonstrated increased cardiotoxicity in mice undergoing alternate-day fasting after Dox administration.

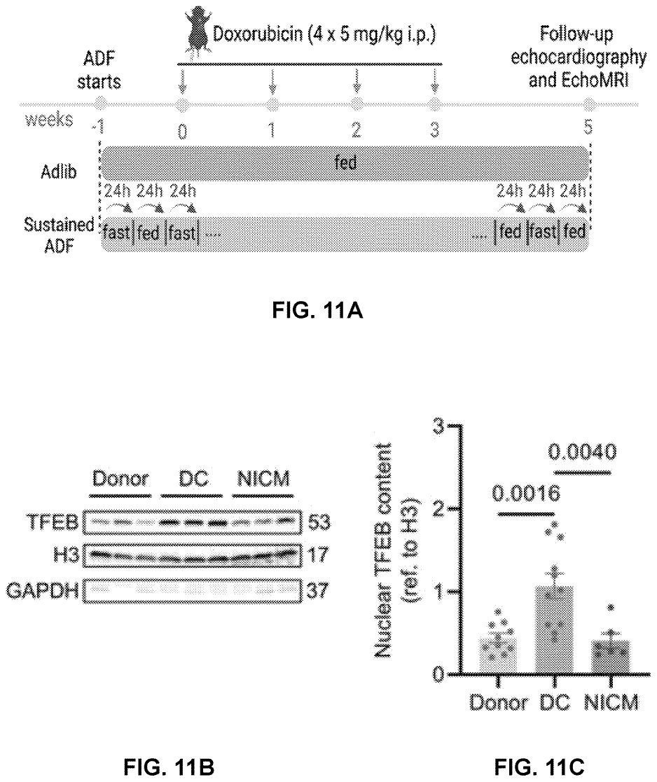

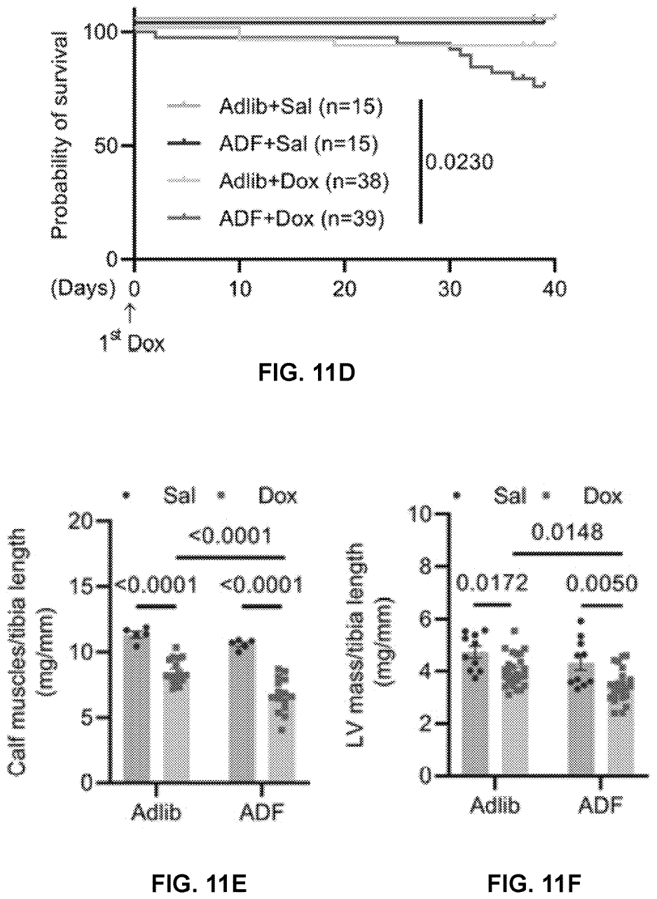

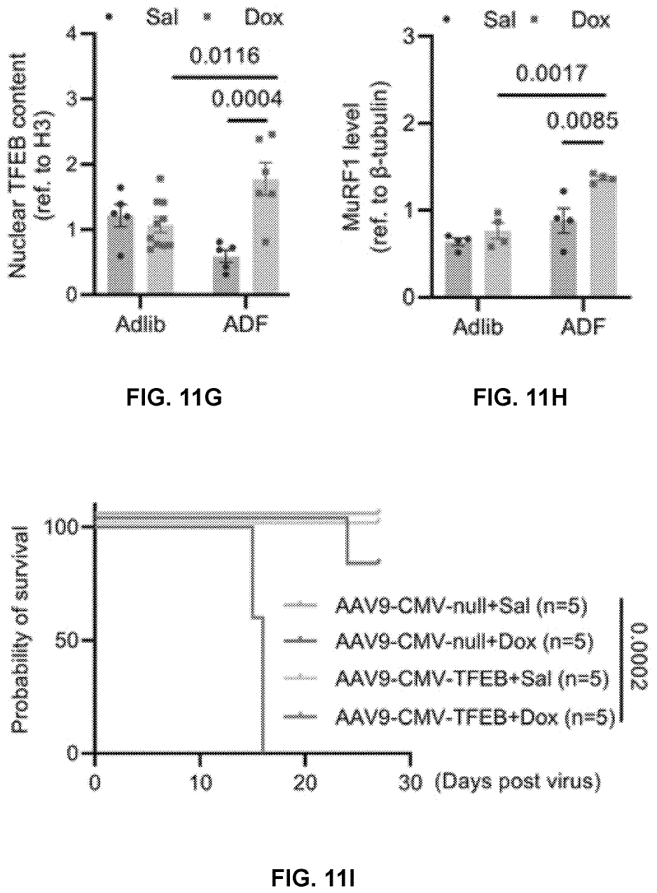

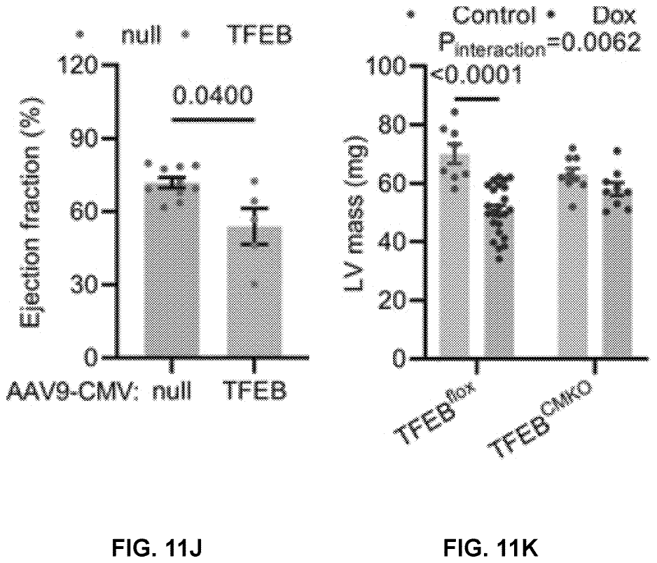

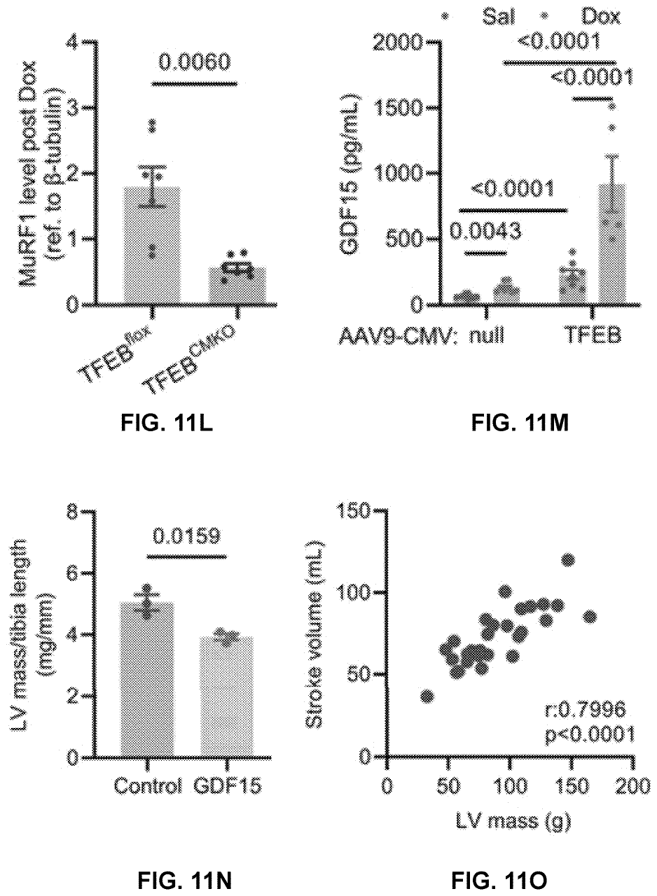

Sustained alternate-day fasting potentiates doxorubicin cardiotoxicity (FIG. 11A). Dox decreases cardiac muscle mass via MuRF1, and ADF increases Transcription Factor EB (TFEB) which activates MuRF1. This suggests that ADF and nuclear TFEB potentiate doxorubicin cardiotoxicity (FIG. 11B, FIG. 11C, FIG. 11D, FIG. 11E, FIG. 11F, FIG. 11G, FIG. 11H, FIG. 11I, FIG. 11J, FIG. 11K, FIG. 11L, FIG. 11M, FIG. 11N, FIG. 11O).

### Example 3: Alternate Day Fasting after Dox Activates UCP-1 and SLIT2

To characterize the activation of UCP-1 and SLIT2 during ADF after Dox administration, the following experiments were conducted. Interscapular brown adipose tissue samples from the mice described in Example 1 were subjected to immunoblots to quantify and compare concentrations of phosphorylated mTOR (p-mTOR) and SLIT2 in the Adlib DOX and ADF post-Dox treatment groups. ADF after Dox resulted in increased p-mTOR and UCP1 in interscapular brown adipose tissue. Knockout of UCP1 attenuated Dox cardiotoxicity demonstrated by a small change in body weight, an increased heart weight to body weight ratio, and increased left ventricular mass to body weight ratio. UCP1 knockout mice were compared to wildtype controls treated with Dox (5 mg/kg i.p.×3 doses).

Treatment with recombinant SLIT2 potentiated Dox cardiotoxicity while neutralizing SLIT2 attenuated ADF Dox cardiotoxicity. Administering recombinant SLIT2 during ADF after Dox exacerbated the loss of calf muscles and further decreased left ventricular ejection fraction. Conversely, the neutralization of STIL2 with an anti-SLIT2 antibody attenuated cardiotoxicity, as demonstrated by reduced 10 changes in the ejection fraction, LV mass, EDV, and fractional shortening. Additionally, SLIT2 neutralization attenuated fasting-induced BAT expansion.

What is claimed is:

1. A method of preventing, reducing, or reversing a chemotherapy-induced cardiotoxicity in a subject in need thereof, the method comprising administering to the subject a therapeutically effective amount of an inhibitor configured to inhibit brown adipose tissue signaling or expansion as an adjuvant to a chemotherapy.

2. The method of claim 1, wherein the inhibitor targets a protein or a gene within a SLIT2 signaling pathway, wherein the SLIT2 signaling pathway facilitates the brown adipose tissue signaling or expansion.

3. The method of claim 2, wherein the SLIT2 signaling pathway comprises UCP1, SLIT2, ROBO1, ROBO2, ROBO3, ROBO4, mTOR, TFEB and any combination thereof.

4. The method of claim 3, wherein the inhibitor comprises an anti-SLIT2 antibody configured to bind to SLIT2 and inhibit the SLIT2 signaling pathway.

5. The method of claim 4, wherein the anti-SLIT2 antibody is administered to the subject in at least two doses.

6. The method of claim 5, wherein the anti-SLIT2 antibody is administered to the subject in 6 doses.

7. The method of claim 4, wherein the therapeutically effective amount of the anti-SLIT2 antibody comprises a dose of 0.3 mg of anti-SLIT2 antibody/kg body mass.

8. The method of claim 1, wherein treating the chemotherapy-induced cardiotoxicity comprises maintaining a baseline percentage of brown adipose tissue, the baseline percentage of brown adipose tissue comprising the percentage of the subject before initiation of the chemotherapy.

9. The method of claim 8, wherein the baseline percentage of brown adipose tissue comprises the percentage of brown adipose tissue present in a subject before fasting or administration of a chemotherapy agent.

10. The method of claim 1, wherein the chemotherapy is doxorubicin.

11. The method of claim 1, wherein treating the chemotherapy-induced cardiotoxicity further comprises maintaining the subject on a non-fasting diet.
